(19) United States
(12) Patent Application Publication
(10) Pub. No.: US 2022/0040304 A1
(43) Pub. Date: Feb. 10, 2022

(54) A COMPOSITION FOR CANCER CELL DEATH AND ITS USE

(71) Applicant: Industry-University Cooperation Foundation Hanyang University, Seoul (KR)

(72) Inventors: Young-Pil KIM, Seoul (KR); Eun Hye KIM, Uiwang-si (KR)

(73) Assignee: Industry-University Cooperation Foundation Hanyang University, Seoul (KR)

(21) Appl. No.: 17/287,818

(22) PCT Filed: Oct. 22, 2019
(86) PCT No.: PCT/KR2019/013900

§ 371 Date: Apr. 22, 2021

(30) Foreign Application Priority Data
Oct. 22, 2018 (KR) 10-2018-0126301

(51) Int. Cl. A61K 41/00; A61K 38/16; A61K 38/44; A61K 31/4985; A61P 35/00; A61K 47/64

(52) U.S. Cl. CPC A61K 41/0057; A61K 38/16; A61K 47/64; A61K 31/4985; A61P 35/00; A61K 38/44

(57) ABSTRACT

The present application relates to a cancer cell death composition and a cancer cell death method. The present application relates to an invention using a mechanism that provides reactive oxygen species to cell membranes of cancer cells, so as to break down the cell membranes of cancer cells, thereby enabling cancer cell death.

### CROSS REFERENCE TO RELATED APPLICATIONS

This application is a National Stage of International Application No. PCT/KR2019/013900 filed Oct. 22, 2019, claiming priority based on Korean Patent Application No. 10-2018-0126301 filed Oct. 22, 2018.

### TECHNICAL FIELD

The present application relates to a composition fora cancer cell death, which comprises a reactive oxygen species (hereinafter referred to as ROS)-generating protein that generates ROS and a protein capable of directly or indirectly binding to the cell membrane of a cancer cell.

The present application relates to a composition fora cancer cell death, which comprises a ROS-generating protein that generates ROS, a protein capable of directly or indirectly binding to the cell membrane of a cancer cell, and a protein for providing a light.

The present application relates to a method for inducing a cancer cell death using a composition for a cancer cell death.

The present application relates to various uses of the composition.

### BACKGROUND ART

Methods for inducing a cancer cell death include a surgical method through surgical incision, a method using radiation, and a method of taking an anticancer drug.

These methods generally not only kill cancer cells, but also have a problem of killing normal cells. In addition, it is difficult for these methods to kill cancer cells deep in the body.

The recently noteworthy method for inducing a cancer cell death is a photodynamic method. The photodynamic method is a method for inducing a cancer cell death using ROS by injecting a photosensitizer that generates the ROS generated by a chemical reaction by a light and an oxygen into the body.

Photosensitizers used in the photodynamic method are chemical photosensitizers. However, these take a long time to decompose and discharge due to slow metabolism in the human body, and are accumulated at a low concentration in normal cells, thereby having a side effect of phototoxicity when exposed to a light. Particularly, since the chemical photosensitizers need a light provided from the outside, there is a limit to a light penetration, and therefore they are not suitable for tumors with a large volume or a cancer cell deep in the body.

Instead of a chemical photosensitizer, a gene encoding a protein generating ROS in response to a light may be directly injected into the body using a vector.

However, since most vectors originate from pathogenic viruses, they have problems in stability and toxicity. In addition, since having no specificity to a cancer cell, there is the possibility that the vectors enter a normal cell, thereby killing not only cancer cells but also a normal cell. Particularly, since a vector containing genes do not act from the step of recognizing a cancer cell, but should go through a process of being expressed as a protein after entering the cell, they need expression time and have a great difference in expression rate from cell to cell, and therefore, even after expression, a ROS release effect may be limited by various causes including an intracellular expression location and an intracellular proteolytic mechanism.

Accordingly, to overcome the problems of the methods for inducing a cancer cell death by ROS release, there is a demand for developing a method of increasing a cancer cell death without affecting a normal cell death and killing a cancer cell deep in the body.

### RELATED ART DOCUMENTS

### Patent Documents

(Patent Document 001) 1. International Patent Publication No. WO2011US052156
(Patent Document 002) 2. International Patent Publication No. WO2018EP066982

### Non-Patent Documents

(Non-patent Document 001) 1. J Photochem Photobiol B. 2018 November; 188:107-115. doi: 10.1016/j.jphotobiol.2018.09.006.
(Non-patent Document 002) 2. Dokl Biochem Biophys. 2018 September; 482(1):288-291. doi: 10.1134/S1607672918050150.
(Non-patent Document 003) 3. Acta Naturae. 2016 October-December; 8(4):118-123.

### DISCLOSURE

### Technical Problem

The present application is directed to providing a composition for a cancer cell death, which comprises a protein for generating reactive oxygen species (ROS) and a protein capable of directly or indirectly binding to a cell membrane of the cancer cell.

The present application is also directed to providing a composition for a cancer cell death, which comprises a protein for generating ROS, a protein capable of directly or indirectly binding to a cell membrane of the cancer cell, and a protein for providing a light.

The present application is also directed to providing a method for inducing a cancer cell death using the composition of a cancer cell death.

The present application is also directed to providing various uses of the composition for a cancer cell death.

To resolve the above-described technical problems, the present application provides ROS to the cell membrane of a cancer cell to destroy the cell membrane of the cancer cell, resulting in a cancer cell death.

Therefore, in one aspect, the present application provides a method for inducing a cancer cell death, comprises:

i) preparing a cancer cell death-fusion protein comprising a first protein for generating reactive oxygen species (ROS); and a second protein for specifically binding to a cell membrane of the cancer cell;

ii) inducing the cancer cell death-fusion protein to be attached to the cell membrane of the cancer cell; and

iii) providing a light to produce ROS by the first protein.

In addition, the present application provides the cancer cell death-fusion protein further comprises a third protein for providing a light. Wherein substrate is provided such that the third protein generate a light.

In addition, the present application provides the step of the iii) providing a light to produce ROS by the first protein; is carried out using a light provided from the outside.

In the method for inducing a cancer cell death, the second protein directly or indirectly binds to the cell membrane of a cancer cell to induce the first protein to be placed near the cell membrane of the cancer cell, and therefore, ROS generated by the reaction between the first protein and a light is provided to the cell membrane of the cancer cell, leading to the cancer cell death.

In another aspect, the present application provides a cancer cell death-fusion protein, comprises:

a first protein for generating reactive oxygen species (ROS); and a second protein for specifically binding to a cell membrane of the cancer cell.

In still another aspect, the present application provides the cancer cell death-fusion protein further comprises a third protein for providing a light.

Particularly, the second protein is a protein having any one function selected from the following functions:

a protein capable of specifically binding to specific receptors expressed on a surface of the cancer cell;

a protein capable of specifically binding to membrane protein constituting the cancer cell membrane;

a protein capable of specifically binding to a ligand which is able to specifically bind to the specific receptor expressed on the surface of the cancer cell, or a ligand which is able to specifically binding to membrane protein constituting a cancer cell membrane;

a protein capable of binding to a specific region of an antibody capable of specifically binding to a protein expressed on the surface of the cancer cell; and

a peptide that has permeability to a cell membrane of the cancer cell.

The present application provides the cancer cell death-fusion protein further comprises at least one of a first linker capable of liking the first protein with the second protein; or a second linker capable of liking the second protein with the third protein.

### Advantageous Effects

According to the present application, the following effects are generated.

First, according to the present application, a composition for a cancer cell death, which comprises a protein for generating ROS and a protein capable of directly or indirectly binding with the cell membrane of a cancer cell, can be provided. Moreover, the composition can provide a composition for a cancer cell death, which further comprises a protein for providing a light.

Second, according to the present application, a method for inducing a cancer cell death using the composition for a cancer cell death can be provided. Particularly, the method of the present application can provide ROS by attaching the composition to the cell membrane of a cancer cell, thereby inducing the cancer cell death. Moreover, this method may provide an effect of not affecting a normal cell death, but only affecting a cancer cell death.

Third, according to the present application, a pharmaceutical composition comprising the composition for a cancer cell death can be provided.

Fourth, according to the present application, according to the present application, various uses using the composition for a cancer cell death can be provided.

### DESCRIPTION OF DRAWINGS

FIG. 1 is a schematic diagram of a fusion protein according to the present application.

FIGS. 2 to 7 are schematic diagrams of plasmid vectors used in the present application.

FIGS. 8A and 8B show electrophoresis results for proteins.

FIG. 9 is a set of graphs showing an absorbance spectrum and a fluorescence spectrum according to proteins. BL represents bioluminescence, and FL represents fluorescence. (a) shows the results for RLuc8.6; RLuc8.6-KR; and KR, respectively, and (b) shows the results for RLuc8; RLuc8-MS; and MS, respectively.

FIG. 10 is a set of graphs showing a bioluminescence spectrum and a fluorescence spectrum according to proteins. (a) shows the results for RLuc8.6; RLuc8.6-KR; and KR, respectively, and (b) shows the results of RLuc8; RLuc8-MS; and MS, respectively.

FIG. 11 is a set of graphs showing the measurement of ROS generated by the reaction of a protein with various concentrations of coelenterazine-h (hereinafter referred to as Co-h). A substrate reaction time is 5 minutes, and the degree of ROS generation is represented by a fluorescence reduction rate (% fluorescence beaching) using dihydroethidium (DHE, a superoxide-measuring chemical reagent, (a))) or anthracene-9,10-dipropionic acid (ADPA, singlet oxygen-measuring chemical reagent, (b)). (a) shows the result for Rluc8.6-KR protein, and (b) shows the result for Rluc8-MS protein.

FIG. 12 is a set of graphs showing ROS measurement according to the reaction time between a protein and Co-h. The concentration of Co-h is 150 μM, and the degree of ROS generation is represented by a fluorescence reduction rate (% fluorescence beaching) using dihydroethidium (DHE, a superoxide-measuring chemical reagent, (a))) or anthracene-9,10-dipropionic acid (ADPA, singlet oxygen-measuring chemical reagent, (b)). (a) shows the result for Rluc8.6-KR protein, and (b) shows the result for Rluc8-MS protein.

FIG. 13 is a set of graphs showing the measurement of ROS generated using various types of proteins without a substrate after light irradiation (10 mW/cm², 30 min). (a) is the result of measuring superoxide by DHE, and (b) is the result of measuring singlet oxygen by ADPA.

FIG. 14 is a set of graphs showing the measurement of ROS generated by the reaction (30-min reaction) of various types of proteins with a 150 μM Co-h substrate without light irradiation. (a) shows the result of measuring superoxide by DHE, and (b) shows the result of measuring singlet oxygen by ADPA.

FIG. 15 is a set of graphs showing the measurement of ROS generated by the reaction (30-min reaction) of proteins Rluc8.6-KR (A) and Rluc8-MS (B) with a 150 μM Co-h substrate after ROS scavenger treatment without light irradiation. (a) shows the result of measuring superoxide by DHE, and (b) shows the result of measuring singlet oxygen by ADPA.

FIGS. 16 and 17 are graphs of confirming the stability of bioluminescence signals of proteins. Proteins were added in the presence of phosphate-buffered saline (PBS) or 100% mouse serum, and then bioluminescence was measured by time. Bioluminescence was measured using 150 μM Co-h, and a relative bioluminescence signal is represented by a change rate to the initial luminescence signal. FIG. 16(a) shows the result for Rluc8.6 protein, FIG. 16(b) shows the result for Rluc8.6-KR-LP protein, FIG. 17(a) shows the result for Rluc8 protein, and FIG. 17(b) shows the result for Rluc8-MS-LP protein.

FIGS. 18 and 19 show cell death according to light irradiation time after MCF-7 breast cancer cell lines are treated with various proteins. Light irradiation was performed under 10 mW/cm², and cell death was measured by the colorimetric change of 3-(4,5-dimethylthiazol-2-yl)-2,5-diphenyltetrazolium bromide (MTT). FIG. 18 shows the results for KR, RLuc8.6-KR and RLuc8.6-KR-LP proteins, respectively. FIG. 18(a) shows the result of measuring the colorimetric change of MTT, and FIG. 18(b) is the graph showing a relative cell viability based on the absorbance of a MTT colorimetric solution. FIG. 19 shows the results for MS, RLuc8-MS and RLuc8-MS-LP proteins, respectively. FIG. 19(a) shows the result of measuring the colorimetric change of MTT, and FIG. 19(b0 is the graph showing a relative cell viability based on the absorbance of a MTT colorimetric solution.

FIGS. 20 and 21 show cell death according to time after treatment with 150 μM Co-h without light irradiation after MCF-7 breast cancer cell lines are treated with proteins (KR, RLuc8.6-KR and RLuc8.6-KR-LP, respectively). FIG. 20(a) shows the result of measuring the colorimetric change of MTT, and FIG. 20(b) is the graph showing a relative cell viability based on the absorbance of a MTT colorimetric solution. FIG. 21 is a set of optical microscope images showing the number of cells attached to a surface after the colorimetric change in MTT solution is measured, a solution in a plate is removed and then the plate is washed with a buffer.

FIGS. 22 and 23 show cell death according to time after treatment with 150 μM Co-h without light irradiation after MCF-7 breast cancer cell lines are treated with proteins (MS, RLuc8-MS and RLuc8-MS-LP, respectively). FIG. 22(a) shows the result of measuring the colorimetric change of MTT, and FIG. 22(b) is the graph showing a relative cell viability based on the absorbance of a MTT colorimetric solution. FIG. 23 is a set of optical microscope images showing the number of cells attached to a surface after the colorimetric change in MTT solution is measured, a solution in a plate is removed and then the plate is washed with a buffer.

FIGS. 24 and 25 show cell death according to light irradiation time after MCF-7 breast cancer cell lines are treated with proteins (KR, RLuc8.6-KR and RLuc8.6-KR-LP, respectively). Light irradiation was performed under the condition of 10 mW/cm². FIG. 24 is a set of fluorescence microscope images showing cell death of KR, Rluc8.6-KR and Rluc8.6-KR-LP, respectively detected with SYTOX Green (dead cell-specific dye, green, indicated by an arrow) and DAPI (live cell-specific dye, blue). FIG. 25 is a graph showing the fluorescence of SYTOX Green obtained from the fluorescence microscope images of FIG. 24 by measuring an average fluorescence of SYTOX green obtained from the fluorescence microscope images based on the same area.

FIGS. 26 and 27 show bioluminescence-based cytotoxic effects in MCF-7 breast cancer cell lines. Cell death according to time after treatment with 150 μM of Co-h without light irradiation was confirmed. FIG. 26 shows fluorescence microscope images comparing cell death of KR, Rluc8.6-KR and Rluc8.6-KR-LP, respectively detected using SYTOX Green (dead cell-specific dye, green, indicated by an arrow) and DAPI (live cell-specific dye, blue). FIG. 27 is a graph showing the measurement of an average fluorescence of SYTOX green obtained from the fluorescence microscope images based on the same area.

FIGS. 28 and 29 show cell death according to light irradiation time after MCF-7 breast cancer cell lines are treated with proteins (MS, Rluc8-MS and Rluc8-MS-LP, respectively). Light irradiation was performed under the condition of 10 mW/cm². FIG. 28 is a set of fluorescence microscope images showing cell death between MS, Rluc8-MS and Rluc8-MS-LP detected with EthD-1 (dead cell-specific dye, red; indicated by an arrow) and DAPI (live cell-specific dye, blue). FIG. 29 is a graph showing the measurement of an average fluorescence of EthD-1 obtained from the fluorescence microscope images based on the same area.

FIGS. 30 and 31 show bioluminescence-based cytotoxic effects in MCF-7 breast cancer cell lines. Cell death according to time after treatment with 150 μM of Co-h without light irradiation was confirmed. FIG. 30 shows fluorescence microscope images showing cell death of MS, Rluc8-MS and Rluc8-MS-LP, respectively detected using EthD-1 (dead cell-specific dye, red; indicated by an arrow) and DAPI (live cell-specific dye, blue; indicated by an arrow). FIG. 31 is a graph showing the measurement of an average fluorescence of EthD-1 obtained from the fluorescence microscope images based on the same area.

FIGS. 32 and 33 show the cytotoxic effects according to time with a protein probe (RLuc8.6-KR-LP) in MCF-7 breast cancer cell lines. The fluorescence image of cells which are treated with the protein probe (10 μM) in a cell culture solution at 37° C. for 24 hours, subjected to a bioluminescence reaction (FIG. 32) and LED light irradiation (FIG. 33), incubated over time, and then stained with SYTOX Green (indicated by an arrow) and DAPI. FIG. 32 shows fluorescence images of cells over time after treated with 150 μM Co-h for 5 minutes. FIG. 33 shows fluorescence images of cells over time after exposure to light for 1, 5 and 10 minutes by light irradiation at 10 mW/cm².

FIG. 34 shows the result of analyzing the cytotoxic effects over reaction time after MCF-7 breast cancer cell lines treated with RLuc8.6-KR-LP protein (10 μM). The results obtained under conditions of fetal bovine serum (FBS)-free media (RPMI; top) and FBS-containing media (bottom) were compared at the same time. The fluorescence images of cells are obtained by staining the cells incubated over time and stained with SYTOX Green (indicated by an arrow) and DAPI.

FIG. 35 shows the result of analyzing the cytotoxic effects at different concentrations of RLuc8.6-KR-LP protein in MCF-7 breast cancer cell lines. The fluorescence images of cells are obtained by adding the protein (RLuc8.6-KR-LP) by concentration to FBS-free (top) and FBS-containing (bottom) RPMI media, maintaining it for 12 hours, and treating 150 μM Co-h, SYTOX Green (indicated by an arrow) and DAPI at the same time.

FIG. 36 shows the result obtained by fluorescence-activated cell sorting (FACS) showing protein probe binding and cytotoxic effects induced by bioluminescence in MCF-7 breast cancer cell lines. FIG. 36 shows the FACS results for non-treated cells, Rluc8.6-KR-treated cell and Rluc8.6-KR-LP-treated cell (first row), and then the FACS results for the cell treated with SYTOX Green 24 hours (second row), and the cell treated with DAPI (third row) after treatment with 150 μM Co-h.

FIG. 37 is a set of graphs showing the flow cytometric analysis result of FIG. 36, represented by a rate of the number of cells showing fluorescence with respect to the total number of cells.

FIG. 38 is a set of fluorescence images of cells, showing the cytotoxic effect of RLuc8.6-KR-LP protein by light irradiation in various breast cancer cell lines (MCF-7, BT-474, MDA-MB-435, SK-BR-3, MDA-MB-231 and MCF-10A, respectively). Protein probes were treated with Rluc8.6-KR (top in comparative image) or Rluc8.6-KR-LP (bottom in comparative image) under the same conditions (final 10 μM, 12 hrs, serum-free media), and subjected to light irradiation (10 mW/cm², 10 min). Afterward, fluorescence images were obtained by adding SYTOX Green and maintaining the probes for 30 minutes, and treating DAPI for five more minutes. SYTOX Green (indicated by an arrow)- and DAPI-stained fluorescence images were superimposed, and compared at low magnification (×200, top) and high magnification (×800, bottom).

FIGS. 39 and 40 show fluorescence images of cells exhibiting the bioluminescence-based cytotoxic effect of RLuc8.6-KR-LP protein in various breast cancer cell lines (MCF-7, BT-474, MDA-MB-435, SK-BR-3, MDA-MB-231 and MCF-10A, respectively). LP-free and LP-binding protein probes were treated under the same conditions (final 10 μM, 24 hrs, FBS-free media), and treated with Co-h (150 μM, 5 min). Afterward, SYTOX Green was added, and the cells were maintained for 30 minutes, thereby obtaining fluorescence images, and DAPI was then additionally treated for 5 minutes, thereby obtaining fluorescence images (red: EthD-1; indicated by an arrow, green: SYTOX Green; indicated by an arrow, and blue: DAPI). FIG. 39 shows the result of comparing Rluc8.6-KR and Rluc8.6-KR-LP, and FIG. 40 shows the result of comparing Rluc8-MS and Rluc8-MS-LP.

FIG. 41 shows fluorescence images of cells exhibiting a bioluminescence-based cytotoxic effect on a cancer cell lines (primary cells) extracted from breast cancer patients. The breast cancer cell lines are triple negative malignant breast cancer cell lines in which an estrogen receptor, a progesterone receptor and HER2 are not expressed, and protein probes were treated with Rluc8.6-KR and Rluc8.6-KR-LP, respectively in final 10 μM primary cell culture media for 24 hours, Co-h (150 μM) was treated for 5 minutes, or LED light irradiation was performed at 10 mW/cm² for 5 minutes. Afterward, fluorescence images were obtained by treating SYTOX Green (indicated by an arrow) and DAPI, superimposed and compared.

FIGS. 42 to 44 show the results obtained by mouse imaging, showing the bioluminescence-based cytotoxic effect of RLuc8.6-KR-LP protein in a breast cancer cell line (MDA-MB-231), and tissue sizes. LP-binding protein probes were intratumorally treated under the same conditions (final 10 μM, 24 hrs), and Co-h (150 μM) was subcutaneously injected. FIG. 42 is a set of images obtained by an IVIS spectrum (Xenogen Inc.), and FIG. 43 shows the sizes of breast cancer tissue. FIG. 44 is a graph obtained by measuring the sizes of breast cancer tissue per date.

### MODES OF THE INVENTION

The present application is characterized by using a mechanism in which reactive oxygen species (ROS) are provided to the cell membrane of a cancer cell to destroy the cell membrane of the cancer cell, thereby inducing the cancer cell death.

A cancer cell death may occur by various mechanisms such as apoptosis, necrosis and autophagy, and the mechanism of killing cancer cells may vary depending on where a protein affects a cancer cell.

A protein may affect a cancer cell death by affecting various parts such as the mitochondria, ribosomes, endoplasmic reticulum and cell membrane of a cancer cell, but a protein of the present application may affect a cancer cell death by destroy the cell membrane of a cancer cell.

The present application relates to a fusion protein comprising a protein capable of directly or indirectly binding to a cell membrane of the cancer cell and an ROS-generating protein that generates ROS, and a use thereof.

The protein generating ROS using the protein capable of directly or indirectly binding to a cell membrane of the cancer cell may be placed around the cell membrane, and the ROS-generating proteins are activated, thereby inducing the cancer cell death.

The activation of the ROS-generating protein may be achieved by light. For example, light may be provided from the outside using a specific light (e.g., LED, laser, etc.), or may be generated by the fusion protein itself by additionally comprising a protein for providing a light. Particularly, when the protein for providing a light is used, a specific substrate compound that activates the protein for providing a light may be used.

Meanwhile, the protein capable of directly or indirectly binding to a cell membrane of the cancer cell may be selected to use the following methods:

a protein capable of specifically binding to specific receptors expressed on a surface of the cancer cell;

a protein capable of specifically binding to membrane protein constituting the cancer cell membrane;

a protein capable of specifically binding to a ligand which is able to specifically bind to the specific receptor expressed on the surface of the cancer cell, or a ligand which is able to specifically binding to membrane protein constituting a cancer cell membrane;

a protein capable of binding to a specific region of an antibody capable of specifically binding to a protein expressed on the surface of the cancer cell; and

a peptide that has permeability to a cell membrane of the cancer cell.

When such the protein directly or indirectly binds to a cell membrane of the cancer cell, the ROS-generating protein used therewith provides ROS to the cancer cell membrane without being introduced into an interior of the cells due to its size.

Particularly, since the ROS-generating protein generates ROS at the range of approximately 10 to 20 nm around it for a short period of time (approximately 0.01 μs), the ROS may be provided effectively to the cancer cell membrane by the protein directly or indirectly binding with the cancer cell membrane, thereby exhibiting an excellent cytotoxic effect on a cancer cell.

Since the present application having such a technical characteristic can selectively a cancer cell death by providing ROS to the cancer cell membrane, the specificity and selectivity for a cancer cell may be remarkably increased. Moreover, after the cancer cell death, in the case of living tissue, the fusion protein of the present application is rapidly degraded and does not accumulate in the body. Therefore, compared with the conventional art in which a gene is introduced into cells via a vector, the present application can improve internal stability as well as solve the problem such as normal cell death occurring when introduced into a normal cell.

Hereinafter, the composition for a cancer cell death of the present application, which has the above-described technical characteristics, and a use thereof will be described in detail.

1. Composition for a Cancer Cell Death

One aspect of the present application relates to a composition for a cancer cell death.

The cancer cells may be skin cancer cell, breast cancer cell, uterine cancer cell, lung cancer cell, liver cancer cell, gastric cancer cell, colon cancer cell, pancreatic cancer cell, blood cancer cell and cancer stem cell thereof, but the present application is not limited thereto.

The composition is a composition which recognizes a cancer cell and is attached to the cell membrane of the cancer cell to provide reactive oxygen species (ROS), thereby inducing the cancer cell death.

The composition of the present application may be a cancer cell death-fusion protein, which comprises a first protein for generating ROS and providing ROS to the cell membrane of a cancer cell; and

a second protein for capable of specifically binding to a cell membrane of the cancer cell.

The “fusion protein” used herein refers to a protein in which two or more different proteins are linked. For example, a fusion protein comprising A protein and B protein is interpreted to include both i) a fusion protein linking between A protein and B protein using a linker; and ii) a fusion protein directly linking between A protein and B protein without a linker.

The first protein is a protein having the ability to generate ROS, which is activated by a light to generate ROS.

The ROS is reactive oxygen species, and comprises all chemically-reactive molecules, including an oxygen atom.

For example, the ROS may be superoxide (O₂), hydroxyl radical (HO), singlet oxygen (¹O₂), hydrogen peroxide (H₂O₂), or hypochlorous acid (HOCl), but the present application is not limited thereto.

The first protein may be activated by a light to generate any one or more selected from the ROS, for example, superoxide, hydroxyl radical, singlet oxygen, hydrogen peroxide, and hypochlorous acid.

The first protein may be any one or more selected from KillerRed, MiniSOG, SOPP, FPFB, SuperNova, mKate2, and KillerOrange, but the present application is not limited thereto. In addition, the first protein includes variants of the KillerRed, miniSOG, SOPP, FPFB, SuperNova, mKate2, and Killerorange.

The KillerRed is an Aequorea victoria-derived green fluorescent protein variant with a size of approximately 27 kDa, and known to generate superoxide when irradiated with green light.

The MiniSOG is derived from a LOV domain of Arabidopsis phototropin 2, has a size of approximately 14 kD, and is known to generate singlet oxygen when irradiated with blue light.

The first protein of the present application may include a part or all of the sequence of one selected from KillerRed, miniSOG, SOPP, FPFB, SuperNova, mKate2 and Killerorange.

The sequence of any one or more selected from the KillerRed, miniSOG, SOPP, FPFB, SuperNova, mKate2 and Killerorange may use a known sequence, for example, the sequence of one disclosed in known databases.

For example, the KillerRed may include a partial or full length of the sequence, that is

(SEQ ID NO: 1)
MLCCMRRTKQVEKNDEDQKISEGGPALFQSDMTFKIFIDGEVNGQKFTIV
ADGSSKFPHGDFNVHAVCETGKLPMSWKPICHLIQYGEPFFARYPDGISH
FAQECFPEGLSIDRTVRFENDGTMTSHHTYELDDTCVVSRITVNCDGFQP
DGPIMRDQLVDILPNETHMFPHGPNAVRQLAFIGFTTADGGLMMGHFDSK
MTFNGSRAIEIPGPHFVTIITKQMRDTSDKRDHVCQREVAYAHSVPRITS
AIGSDED.

The MiniSOG may include a partial or full length of the sequence, that is,

(SEQ ID NO: 2)
MEKSFVITDPRLPDNPIIFASDGFLELTEYSREEILGRNGRFLQGPETDQ
ATVQKIRDAIRDQREITVQLINYTKSGKKFWNLLHLQPMRDQKGELQYFI
GVQLDG.

The first protein of the present application provides the ROS to a cell membrane of the cancer cell by activating with a light, thereby inducing the cancer cell death.

Meanwhile, the second protein is a protein which directly or indirectly binding to a cell membrane of the cancer cell.

The second protein may have any one function selected from the following functions:

a protein capable of specifically binding to specific receptors expressed on a surface of the cancer cell;

a protein capable of specifically binding to membrane protein constituting the cancer cell membrane;

a protein capable of specifically binding to a ligand which is able to specifically bind to the specific receptor expressed on the surface of the cancer cell, or a ligand which is able to specifically binding to membrane protein constituting a cancer cell membrane;

a protein capable of binding to a specific region of an antibody capable of specifically binding to a protein expressed on the surface of the cancer cell; and

a peptide that has permeability to a cell membrane of the cancer cell.

The second protein may be an antibody, artificial antibody, peptide or aptamer which targets a cancer cell, but the present application is not limited thereto. In the present application, the second protein may be a single compound targeting a cancer cell or a protein binding with the compound.

In one embodiment, the second protein may be a protein that capable of specifically binding to specific receptors expressed on a surface of the cancer cell. Here, wherein the second protein may recognize cancer cells expressing the binding specific receptor.

In Table 1, specific receptors, types of a cancer cell expressing the same, and specific peptides as a second protein binding thereto are listed. These are merely examples and the present application is not limited thereto.

[TABLE 1]

| | Second protein | Target | |
|---|---|---|---|
| No. | (Peptide) | receptor | Cancer cells |
| 1 | DHLASLWWGTEL | GPC3 | hepatocellular carcinoma cell HepG2 |
| 2 | NYSKPTDRQYHF | PD-Ll | colon cancer cell line CT26 |
| 3 | IPLPPPSRPFFK | PDGFRβ | human pancreatic carcinoma cell line |
| | | | BxPC3, |
| | | | human breast cancer cell line MCF7 |
| 4 | LMNPNNHPRTPR | PKCδ | Human glioblastoma astrocytoma U373 |
| 5 | [symbol] HHNLTHA [symbol] | PTPRJ | Human cervical cancer cell HeLa, |
| 6 | [symbol] LHHYHGS [symbol] | | Human umbilical vein endothelial cell |
| | | | HUVEC |
| 7 | SPRPRHTLRLSL | TfR 1 | Human liver cancer cell line |
| | | | (SMMC-7721) |
| 8 | TMGFTAPRFPHY | Tie 2 | Human lung adenocarcinoma cell line |
| | | | SPC -A1, |
| | | | Human non-small lung carcinoma cell |
| | | | line H1299 |
| 9 | RMWPSSTVNLSAGRR | CD-21 | Malignant B cell lymphoma |
| 10 | NGYEIEWYSWVTHGMY | VEGFRI (Flt-1) | Primary human cerebral endothelial |
| | | | cells (HCECs) |
| 11 | FRSFESCLAKSH | IL-10 RA | — |
| 12 | YHWYGYTPQNVI | EGFR | — |
| 13 | FCDGFYACYADV | HER2 | Human breast cancer cells (SK-BR-3 |
| | | | and MDA-MB-231) |
| 14 | RGD4C | ανβ3 integrin | Human glioblastoma cells U87MG, |
| | | | Human breast cancer cells MDA-MB-435, |
| | | | Rat glioma cells C6, Mouse fibroblast |
| | | | cells L929 |
| 15 | Cyclic(RGDfK) | ανβ3 integrin | Human non-small lung carcinoma cells |
| | | | H1299, |
| | | | Murine melanoma cells B16-F10, |
| | | | Human embryonic kidney cells HEK-293 |
| 16 | QWAVGHL-Y(CH2-NH)- | Bombesin | Human pancreatic cancer cells CFPAC-1, |
| | L-NH2 | | Human lung cancer cells DMS-53, |
| | | | Human prostate cancer cells PC-3, |
| | | | Human gastric cancer cells MKN-45 |
| 17 | TFFYGGSRGKRNNFKTEEY | Low-density | Glioblastoma (U87 MG), |
| | | lipoprotein | Hepatocarcinoma (SK-Hep-1), Lung |
| | | receptor (LDLr) | carcinoma (NCI-H460) |

For example, the peptide having the sequence of DHLASLWWGTEL in Table 1 may bind with a GPC3 receptor specifically expressed by the hepatocellular carcinoma cell HepG2.

That is, in the fusion protein for a cancer cell death, which comprises the first protein of the present application and the peptide having the sequence of DHLASLWWGTEL (second protein), as the peptide having the sequence of DHLASLWWGTEL binds with the GPC3 receptor, the first protein is placed around the cancer cell membrane of the hepatocellular carcinoma cells HepG2 and activated by a light to provide ROS to the cancer cell membrane, resulting in the selective death of HepG2.

In another example, the peptide having the sequence of SPRPRHTLRLSL in Table 1 may bind with a TfR 1 receptor to selectively kill a human liver cancer cell line (SMMC-7721) through the same mechanism as described above.

In another embodiment, the second protein may be a protein capable of specifically binding to membrane protein constituting the cancer cell membrane.

In one example, the second protein may be a protein capable of specifically binding to membrane protein of certain types of a cancer cell.

In another example, the second protein may be a protein capable of specifically binding to membrane protein specific membrane protein of a cancer cell.

In one embodiment of the present application, as a second protein, SEQ ID NO: 5 (WXEAAYQRFL—here, X may be one selected from A, R, N, D, C, Q, E, G, H, I, L, K, M, F, P, S, T, W, Y and V) is a peptide capable of specifically binding to membrane protein of certain types of a cancer cell, was used. For example, the peptide may be a peptide having a sequence of SEQ ID NO: 6 (WLEAAYQRFL).

The peptide represented by SEQ ID NO: 5 is known to recognize a membrane protein present in the cell membrane of a neuroblastoma cell line such as WAC-2, SH-EP or TET21N and a breast cancer cell line such as MDA-MB-435, MDA-MB-231 or MCF-7, and due to such a characteristic, it is known as a peptide specific to the WAC-2, SH-EP, TET21N, MCF-7, MDA-MA-435 or MDA-MB-231 cell line (Zhang, J. B et al, Cancer Lett 171, 153-164 (2001); Ahmed, S et al, Anal Chem 82, 7533-7541 (2010)).

In Examples 22 to 24 of the present application, the experimental results for various breast cancer cell lines (MCF-7, BT-474, MDA-MB-435, SK-BR-3, MDA-MB-231 and MCF-10A) using SEQ ID NO: 6 (WLEAAYQRFL) are described. Through the results, it can be confirmed that specific breast cancer cell lines such as MCF-7, MDA-MA-435 and MDA-MB-231, among various breast cancer cell lines, were specifically recognized and killed. However, it can be confirmed that cancer cell lines such as BT-474, SK-BR-3 and MCF-10A, which are not recognized by the peptide of SEQ ID NO: 6, were not killed.

Accordingly, according to the example of the present application, it was able to be confirmed that the peptide of SEQ ID NO: 6 specifically binds with a membrane protein of the cell membrane of a breast cancer cell line such as MCF-7, MDA-MA-435 or MDA-MB-231 to place the first protein near the cell membrane of the cancer cell, and the first protein is activated by a light to provide ROS to the cancer cell membrane, thereby inducing the cancer cell death.

In still another embodiment, the second protein may be a protein capable of specifically binding to a ligand which is able to specifically binding to membrane protein constituting a cancer cell membrane.

Types of ligands specifically binding to membrane proteins of specific cancer cells, and receptor proteins as second proteins capable of binding with specific ligands are listed in Table 2. These are merely examples and the present application is not limited thereto.

TABLE 2

| | Second | | |
|---|---|---|---|
| | protein | | |
| No. | (Receptor) | Ligand target | Cancer |
| 1 | Transferrin | TfR ligand (7pep) | Breast |
| | | Transferrin | Breast, Glioma |
| | | Transferrin + TRAIL | Colon |
| | | Transferrin + folate | Glioma |
| | | T7 peptide + TAT | Glioma |
| | | TfR mAb | Glioma |
| 2 | Folate | Folic acid | Lung, Cervical |
| | | Folate | Cervical, Breast, |
| | | | Carcinoma |
| | | Folate + RGD | Carcinoma |
| | | Folate + Asp8 | Breast metastasis |
| | | Folate + transferrin | Glioma |
| 3 | αvβ3 | RGD | Endothelial, |
| | Integrin | | Glioma, Lung, |
| | | | Melanoma, Breast |
| | | RGD + pHA | Glioma |
| | | RGD + Estrone | Breast |
| | | RGD + YPSMA-1 mAb | Prostate |
| | | RGD + Folate | Carcinoma |
| 4 | PSMA | A10 PSMA Apt | Prostate |
| | | YPSMA-1 mAb + RGD | Prostate |
| | | anti-PSMA + anti-CD14 mAb | Prostate |
| 5 | HER2 | Trastuzumab | Breast |
| | | anti-HER2 scFv | Breast |
| | | neu peptide | Breast |
| | | (FCDGFYACYADV) | |
| | | KCCYSL (P6.1 peptide) | Breast |
| 6 | Estrogen | Estrone | Breast |
| | | Estrone + RGD | Breast |
| | | 17β-Estradiol | Breast |
| | | Tamoxifen | Breast |
| 7 | CXCR4 | LFC131 peptide | Lung, Breast |
| | | anti-CXCR4 mAb | Breast |
| | | Peptide R | Lung |
| 8 | ICAM1 | anti-ICAM1 mAb | Breast |
| | | LFA-1 | Cervical |
| 9 | Androgen | Testosterone | Prostate |
| | | α- & β-Bicalutamide | Prostate |
| 10 | CD | CD14) anti-CD14 mAb + | Prostate |
| | | anti-PSMA | |
| | | CD22) anti-CD22 mAb | Lymphoma |
| | | CD44) Hyaluronic acid | Breast, Melanoma |
| | | CD133. Aptamer | Bone |
| 11 | EGFR | anti-EGFR | Breast, Lung |
| | | EGF | Oral |
| | | Cetuximab | Pancreatic |
| 12 | IL | IL4) AP1 peptide | Colon, Glioma |
| | | IL4) Pep-1 | Lung |
| | | IL13) IL13 | Glioma |
| 13 | TNF | TRAIL + Transferrin | Colon |
| 14 | Glycyrrhetinic | glycyrrhetinic acid | Liver |
| 15 | VEGF | anti-VEGF mAb | Pancreatic |
| | | AR7 + T7 peptide | Glioma |

For example, the transferrin protein in Table 2 may specifically bind with the TfR ligand (7pep).

That is, in a cancer cell death-fusion protein, which comprises the first protein of the present application and the transferrin protein (second protein), the transferrin specifically binds with the TfR ligand (7pep) to place the first protein near a cancer cell membrane of the breast cancer cells, and the first protein is activated by a light to provide ROS to the cancer cell membrane, thereby selectively inducing breast cancer cell death.

In another example, the folate protein in Table 2 may bind with folic acid to selectively kill lung cancer cells using the same mechanism as described above.

In yet another embodiment, the second protein may be a protein capable of binding to a specific region of an antibody capable of specifically binding to a protein expressed on the surface of the cancer cell.

As a second protein, each peptide recognizing a specific region (Fc region) of an antibody targeting a cancer cell is listed in Table 3. It is merely an example and the present application is not limited thereto.

[TABLE 3]

| | Second protein | |
|---|---|---|
| No. | (Peptide) | Target |
| 1 | RRGW | Fc region of IgG |
| 2 | HWRGWV | Fc region of IgG |
| 3 | HYFKFD | Fc region of IgG |
| 4 | HFRRHL | Fc region of IgG |
| 5 | NKFRGKYK | Fc region of IgG |

For example, the peptide having the sequence of RRGW in Table 3 may bind with an IgG Fc region of an antibody targeting a cancer cell.

That is, a cancer cell death-fusion protein, which comprises the first protein of the present application and the RRGW sequence (second protein), binds with the Fc region of IgG of the antibody that can specifically bind with a specific protein expressed on a surface of the cancer cell to place the first protein around the cell membrane of the cancer cell, and the first protein is activated by light to provide ROS to the cancer cell membrane, thereby selectively inducing the a cancer cell death.

The antibody targeting a cancer cell may be an antibody that is able to target an epidermal growth factor receptor (EGFR) or an epidermal growth factor receptor (HER2), but the present application is not limited thereto.

For example, an antibody targeting EGFR may be cetuximab or panitumumab, but the present application is not limited thereto.

For example, an antibody targeting HER2 may be trastuzumab, but the present application is not limited thereto.

A cancer cell death-fusion protein according to the present application, which comprises the second protein as described above, may be applied with an immuno-oncology agent if it is used, thereby improving a cytotoxic effect on cancer cells.

In another aspect, a cancer cell death-fusion protein of the present application, further comprises a third protein for providing a light to produce ROS by the first protein.

The third protein for providing a light may be any one selected from a protein capable of providing a light through fluorescence resonance energy transfer (FRET) and a protein capable of providing a light through bioluminescence resonance energy transfer (BRET).

The resonance energy transfer refers to a phenomenon in which resonance energy generated between a donor molecule and an acceptor molecule is transferred. The FRET uses a fluorescent material as a donor, and the BRET uses a bioluminescent material as a donor.

The third protein by the FRET may be a green fluorescent protein (GFP), a yellow fluorescent protein (YFP), a red fluorescent protein (RFP), a blue fluorescent protein (BFP), or a cyan fluorescent protein (CFP), but the present application is not limited thereto.

The third protein of the present application may be any one selected from GFP, YFP, RFP, BFP and CFP.

The third protein by the BRET may be a third protein including a luciferase sequence, but the present application is not limited thereto.

The luciferase refers to an oxidase which oxidizes a substrate to induce bioluminescence.

The luciferase may be Photobacteria luciferase, Firefly luciferase, Railroad worm luciferase, Renilla luciferase, Gaussia luciferase, Metridia luciferase, Cypridiana luciferase, or Oplophorus luciferase (Nanoluc™), but the present application is not limited thereto.

The third protein of the present application may include a part or all of the amino acid sequence encoding any one luciferase selected from Photobacteria luciferase, Firefly luciferase, Railroad worm luciferase, Renilla luciferase (RLuc), Gaussia luciferase, Metridia luciferase, Cypridiana luciferase and Oplophorus luciferase (Nanoluc™).

The luciferase may be wild-type or mutant.

In one embodiment, the third protein of the present application may include a Renilla luciferase sequence.

In another embodiment, the third protein of the present application may include a mutant Renilla luciferase sequence.

For example, the mutant Renilla luciferase (RLuc) may be RLuc8, RLuc8.6, RLuc8 or RLuc6, but the present application is not limited thereto.

The RLuc8 may include a part or all of the sequence

(SEQ ID NO: 3)
MASKVYDPEQRKRMITGPQWWARCKQMNVLDSFINYYDSEKHAENAVIFL
HGNATSSYLWRHVVPHIEPVARCIIPDLIGMGKSGKSGNGSYRLLDHYKY
LTAWFELLNLPKKIIFVGHDWGAALAFHYAYEHQDRIKAIVHMESVVDVI
ESWDEWPDIEEDIALIKSEEGEKMVLENNFFVETVLPSKIMRKLEPEEFA
AYLEPFKEKGEVRRPTLSWPREIPLVKGGKPDVVQIVRNYNAYLRASDDL
PKLFIESDPGFFSNAIVEGAKKFPNTEFVKVKGLHFLQEDAPDEMGKYIK
SFVERVLKNEQ.

The RLuc8.6 may include a part or all of the sequence

(SEQ ID NO: 4)
MASKVYDPEQRKRMITGPQWWARCKQMNVLDSFINYYDSEKHAENAVIFL
HGNATSSYLWRHVVPHIEPVARCIIPDLIGMGKSGKSGNGSYRLLDHYKY
LTAWFELLNLPKKIIFVGHDWGSALAFHYAYEHQDRIKAIVHMESVVDVI
ESWMGWPDIEEELALIKSEEGEKMVLENNFFVETLLPSKIMRKLEPEEFA
AYLEPFKEKGEVRRPTLSWPREIPLVKGGKPDVVQIVRNYNAYLRASDDL
PKLFIESDPGFFYNAIVEGAKKFPNTEFVKVKGLHFLQEDAPDEMGKYIK
SFVERVLKNEQ.

The third sequence using the BRET may be activated by a substrate.

To induce bioluminescence of the third protein using the BRET, the third protein may react with a specific substrate to induce bioluminescence.

The substrate may be luciferin or a luciferin variant (mutant), but the present application is not limited thereto.

The luciferin mutant may be coelentreazine or a coelenterazine derivative, but the present application is not limited thereto.

The coelenterazine derivative may be cp-coelenterazine, f-coelenterazine, coelenterazine-fcp, or coelenterazine-h (Co-h), but the present application is not limited thereto.

For example, in the present application, the substrate may be any one selected from luciferin, coelenterazine, cp-coelenterazine, f-coelenterazine, coelenterazine-fcp, and Co-h.

In one embodiment, in the present application, the substrate may be Co-h.

When the third protein reacts with, for example, oxidizes a substrate, any one of oxygen and adenosine triphosphate (ATP) is needed. This may vary according to the type of luciferase.

For example, the Photobacteria luciferase or Renilla luciferase needs an oxygen when the substrate is oxidized.

In another example, the Firefly luciferase needs an ATP when the substrate is oxidized.

The wavelength of light provided by reacting the third protein with the substrate may vary according to the type of a third protein or a substrate.

For example, the protein including the sequence of the Renilla luciferase generates light having a wavelength ranging from 470 to 480 nm.

In addition, the third protein may bind with a nanoparticle or a polymer, and thus variously adjust the wavelength of a light.

In the present application, a cancer cell death-fusion protein may be comprises a first protein and a second protein. The cancer cell death-fusion protein provides a light provided from the outside so that the first protein generates ROS and the ROS provides the cancer cell membrane, thereby inducing the cancer cell death.

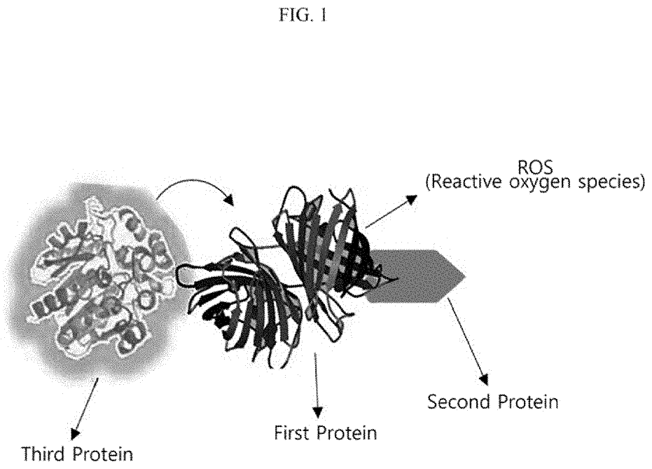

In addition, the cancer cell death-fusion protein may be comprises a first protein, a second protein, and a third protein. The schematic diagram of the cancer cell death-fusion protein of the present application may be shown in FIG. 1. In the cancer cell death-fusion protein, a light may be provided by itself by the third protein such that the first protein may generate ROS, and the ROS may be provided to cell membrane of the cancer cell, thereby inducing the cancer cell death.

The cancer cell death-fusion protein according to the present application may further comprises a linker.

The linker refers to a material having a function of linking a first protein, a second protein and a third protein with each other.

For example, the cancer cell death-fusion protein may comprise the configuration of a first protein-a linker-a second protein.

For example, the cancer cell death-fusion protein may comprise the configuration of a first protein-a second protein-a linker-a third protein.

For example, the cancer cell death-fusion protein may comprise the configuration of a first protein-a first linker-a second protein-a second linker-a third protein.

Here, the first linker and the second linker may be the same or different.

The linker may minimize the potential interference between the first protein, the second protein and the third protein to further increase the cancer cell-killing function of the fusion protein for killing cancer cells. In addition, the linker may increase the structural flexibility of the fusion protein.

The linker may be a functional group of a nucleic acid, an amino acid, a peptide, a polypeptide, a protein or a compound, but as long as one has a function capable of linking the first protein to the third proteins, the present application is not limited thereto.

For example, the functional group may include primary amines, carboxyls, sulfhydryls, carbonyls, and bromide, but the present application is not limited thereto.

The linker may consist of 1 to 100 amino acids, but the present application is not limited thereto.

The amino acids constituting the linker may include hydrophobic amino acids, hydrophilic amino acids, basic amino acids and acidic amino acids, but the present application is not limited thereto.

For example, the hydrophobic amino acids may include valine, leucine, isoleucine, glycine and alanine, but the present application is not limited thereto.

For example, the hydrophilic amino acids may include serine, threonine, tyrosine, proline and asparagine, but the present application is not limited thereto.

For example, the basic amino acids may include lysine, arginine and histidine, but the present application is not limited thereto.

For example, the acidic amino acids may include aspartic acid and glutamic acid, but the present application is not limited thereto.

Specifically, the amino acid sequence may be G, GG, GGG, GGGS, TG, GGGGS, GGGGSTG, GGGGS-SKLTRAETVF or EFGGG, but the present application is not limited thereto (the sequence is in N terminus-to-C terminus direction).

In one embodiment, when domains of the fusion protein are linked using GGG, structural flexibility and stable movement were provided.

In another embodiment, when domains of the fusion protein are linked using EFGGG, structural flexibility and stable movement were provided.

In one embodiment, the cancer cell death-fusion protein may have any one form selected from

RLuc8.6-KillerRed;

RLuc8.6-GGG-KillerRed;

RLuc8.6-KillerRed-WLEAAYQRFL;

RLuc8.6-GGG-KillerRed-WLEAAYQRFL;

RLuc8.6-KillerRed-GGG-WLEAAYQRFL;

RLuc8.6-GGG-KillerRed-GGG-WLEAAYQRFL;

RLuc8-MiniSOG;

RLuc8-GGG-MiniSOG;

RLuc8-EFGGG-MiniSOG;

RLuc8-MiniSOG-WLEAAYQRFL;

RLuc8-GGG-MiniSOG-WLEAAYQRFL;

RLuc8-EFGGG-MiniSOG-WLEAAYQRFL;

RLuc8-MiniSOG-GGG-WLEAAYQRFL;

RLuc8-GGG-MiniSOG-GGG-WLEAAYQRFL; and

RLuc8-EFGGG-MiniSOG-GGG-WLEAAYQRFL.

The cancer cell death-fusion protein may further comprises, optionally, a functional domain, structural domain, or enzymatic domain, which can improve an effect of a cancer cell death, but there is no limit as long as it can have a function capable of increasing an effect of a cancer cell death.

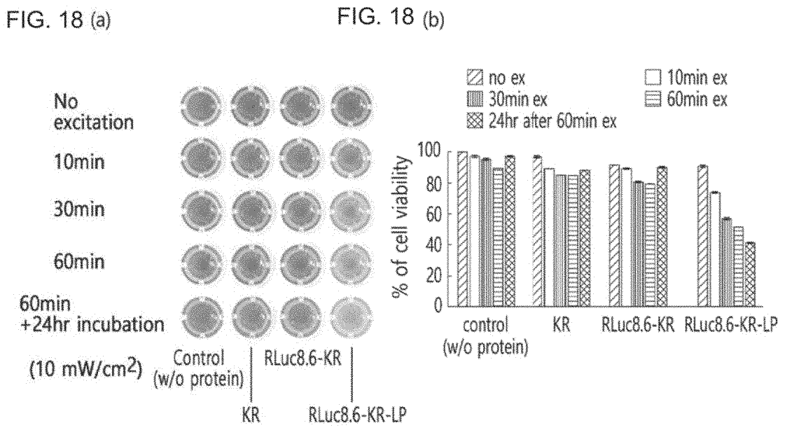

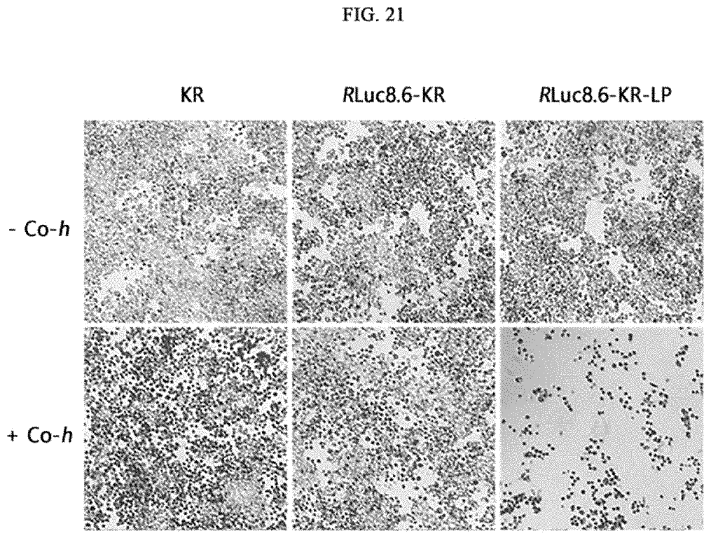

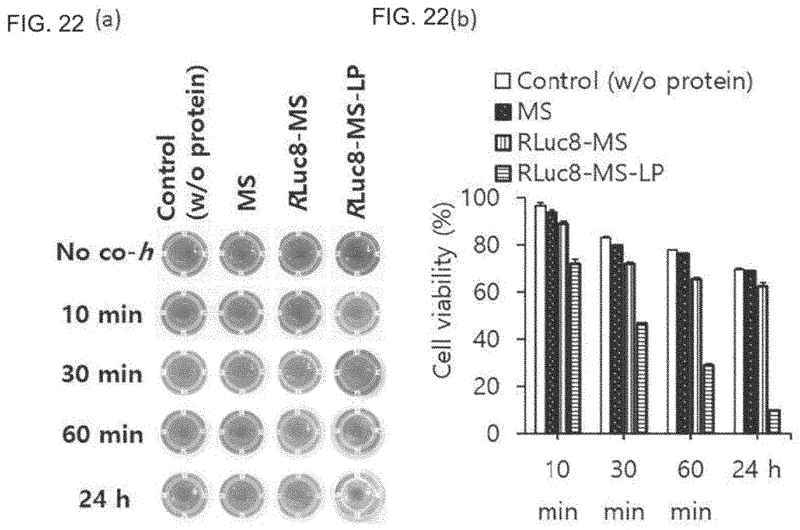

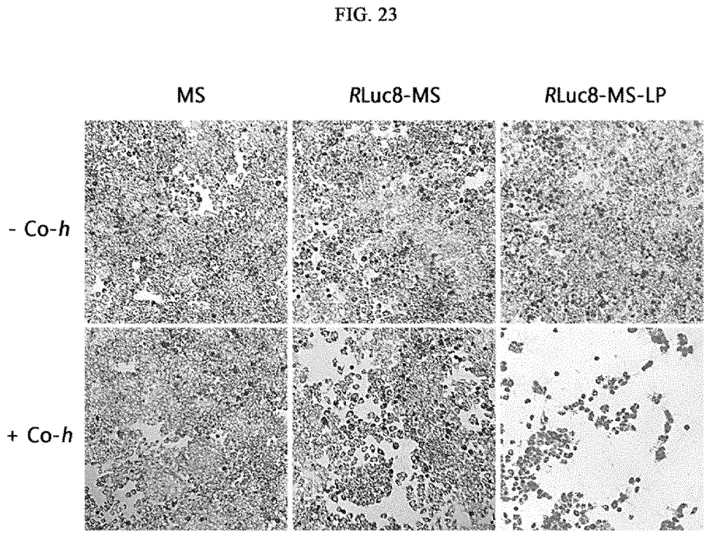

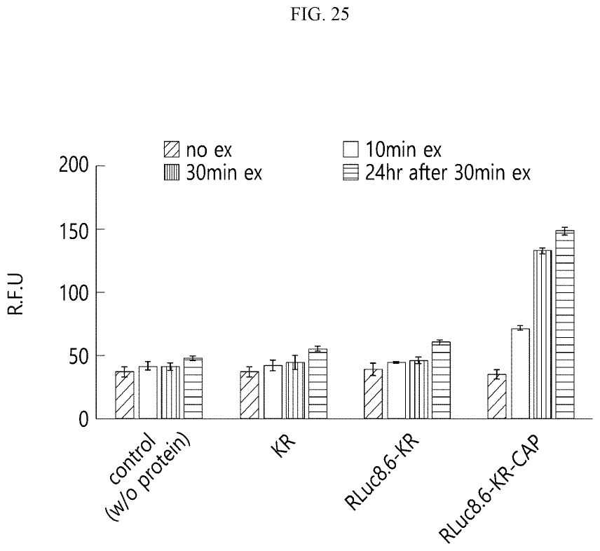

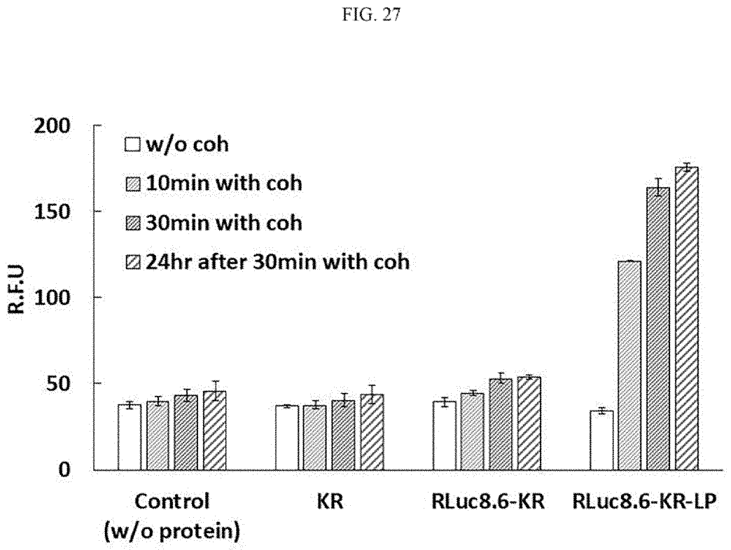

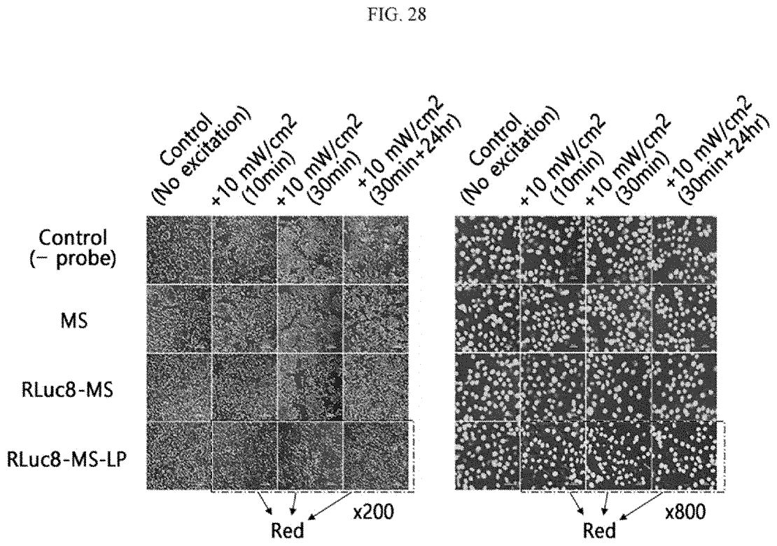

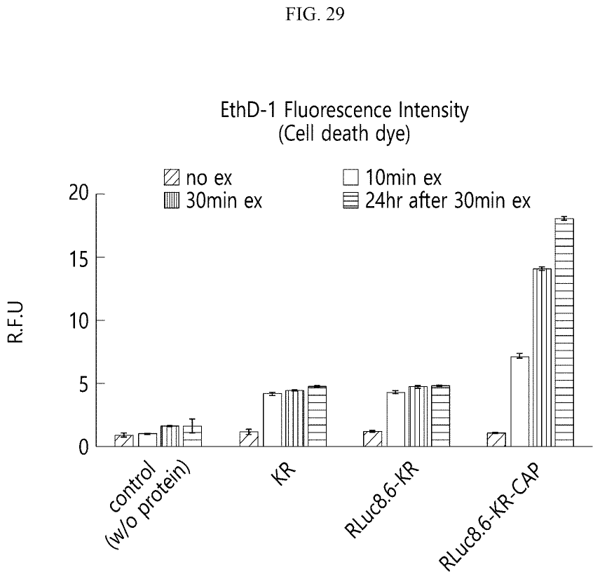

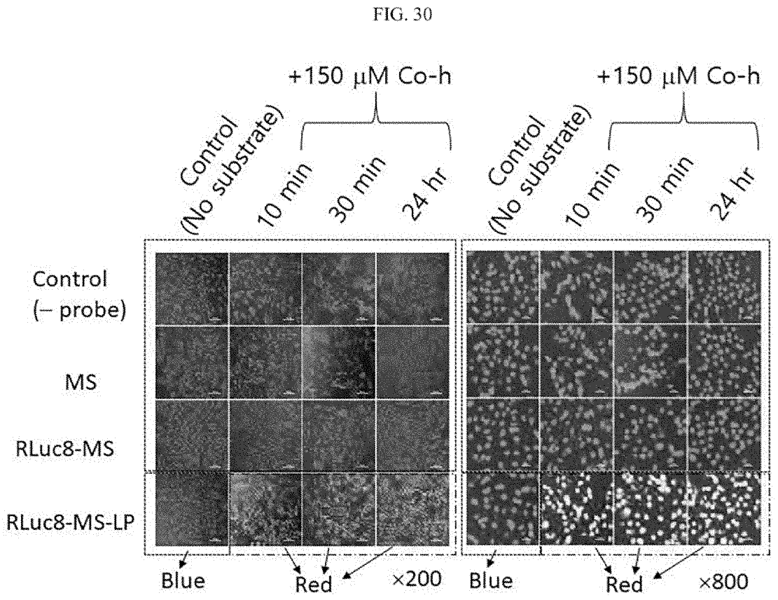

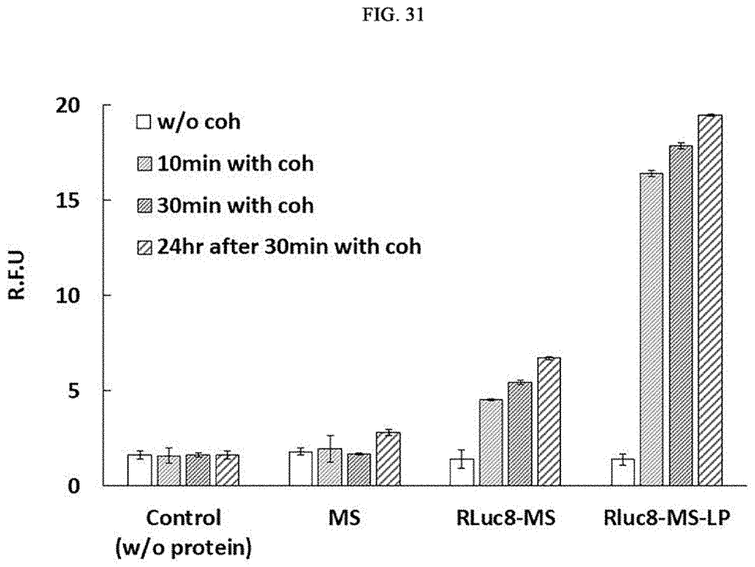

The results for the effect of a cancer cell death of the cancer cell death-fusion protein according to the present application may be confirmed by FIGS. 18 to 31.

FIGS. 18 to 31 show the results confirming the death of breast cancer cells by treating breast cancer cell lines with the cancer cell death-fusion protein.

It can be confirmed that breast cancer cells were killed by ROS generated by activating a first protein by a light provided from the outside as a second protein allowed the first protein to be placed close to the cell membrane of cancer cells by a light provided from the outside irradiation on cells treated with the cancer cell death-fusion protein without Co-h treatment (FIGS. 18, 19, 24, 25, 28 and 29).

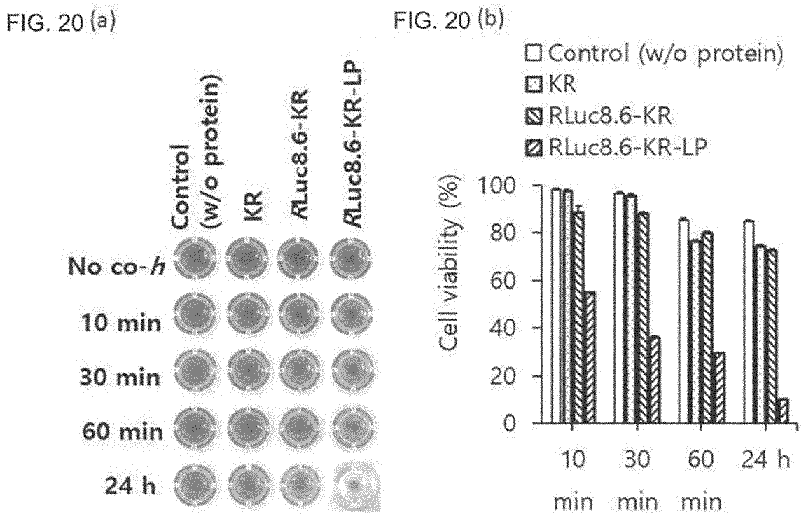

It can be confirmed that breast cancer cells were killed by providing a light from by itself a fusion protein through the reaction between Co-h, which is a substrate, and a third protein without the supply of a light provided from the outside to cells treated with the cancer cell death-fusion protein (FIGS. 20, 21, 22, 23, 26, 27, 30 and 31).

One aspect of the present application relates to a pharmaceutical composition for treating a cancer disease, which comprises the cancer cell death-fusion protein of the present application, and a use thereof.

Here, the “cancer” refers to a disease occurred by cell division continuously progressing without control. The cancer may be a tumor, a neoplasma, a benign tumor, a malignant tumor, carcinoma, or sarcoma, but the present application is not limited thereto. The “cancer cell” used herein is interpreted to mean cells having cancer-causing ability. The term “cancer” or “tumor” is used interchangeably.

The pharmaceutical composition may include a cancer cell death-fusion protein and/or a substrate as active ingredient(s).

Here, the cancer cell death-fusion protein and the substrate have been described above.

The form of the pharmaceutical composition may be suitably selected by one of ordinary skill in the art as needed. For example, the pharmaceutical composition may be used in the form of a solid, gel, gel-spray, or capsule. In addition, the pharmaceutical composition may further comprises an additive such as an excipient, a diluent or a preservative for stability and convenience, but the present application is not limited thereto.

For the treatment of cancer, the pharmaceutical composition may be administered to a subject having a cancer disease, for example, a mammal.

The mammal may include a human, a dog, a cat, a mouse, etc., but the present application is not limited thereto.

The “administration” refers to introduction of the pharmaceutical composition of the present application to a mammal by a suitable method, and an administration route of the pharmaceutical composition of the present application may be a common route that can reach desired tissue. The administration may be oral administration, intraperitoneal administration, intravenous administration, intramuscular administration, subcutaneous administration, endothelial administration, intranasal administration, intrapulmonary administration, intratumor administration, rectal administration, intracavitary administration, intravenous administration, intraperitoneal administration or intrathecal administration, but the present application is not limited thereto.

The pharmaceutical composition may be administered in the form of a protein, not a vector (e.g., a DNA vector encoding the cancer cell death-fusion protein).

The administration of the pharmaceutical composition may be determined by various parameters comprising the type and severity of a cancer disease, the types and contents of an active ingredient and other components contained in the composition, the type of a dosage form, a patient's age, body weight, general health condition, sex and diet, an administration time, an administration route, the excretion rate of the composition, treatment duration, and a co-administered drug.

For example, for an adult, the pharmaceutical composition can be administered into the body at a dose of 50 ml to 500 ml per 1 time, and when the composition is a chemical compound, it may be administered at a dose of 0.1 ng/kg to 10 mg/kg, and if the composition is a monoclonal antibody, it may be administered at a dose of 0.1 ng/kg-10 mg/kg. The administration interval may be once to 12 times a day, and when the administration interval is 12 times a day, the composition may be administered once every two hours.

In addition, the pharmaceutical composition of the present application may be administered by another treatment for improving an immune response, for example, by mixing with an adjuvant or cytokine (or a nucleic acid encoding a cytokine) known in the art.

In addition, the pharmaceutical composition of the present application may be administered alone or in combination with another treatment known in the art, for example, chemotherapy, radiation therapy and surgery, to treat target cancer.

2. Method for Inducing a Cancer Cell Death and Method for Treating Cancer

Another aspect of the present application provides a method for inducing a cancer cell death using the cancer cell death-fusion protein or a composition comprising the same.

In addition, the present application may provide a method for treating cancer, which comprises administering the cancer cell death-fusion protein or a composition comprising the same.

According to the method for inducing a cancer cell death, the second protein selectively recognizes a cancer cell to be attached to the cell membrane of the cancer cell, and the first protein provides ROS generated by a light to the cell membrane of the cancer cell, thereby inducing the cancer cell death.

In one example of the present application,

the method for inducing a cancer cell death described in the present application may comprises:

i) preparing a cancer cell death-fusion protein comprising a first protein for generating reactive oxygen species (ROS); and a second protein for specifically binding to a cell membrane of the cancer cell;

ii) inducing the cancer cell death-fusion protein to be attached to the cell membrane of the cancer cell; and

iii) providing a light to produce ROS by the first protein.

In addition, the cancer cell death-fusion protein may further include a third protein.

The first protein, the second protein and the third protein are the same as described above.

The step of preparing the cancer cell death-fusion protein may be performed by a known method of obtaining a protein.

The step of inducing the cancer cell death-fusion protein to be attached to the cell membrane of a cancer cell is to attach the cancer cell death-fusion protein as close as possible to the cell membrane of the cancer cell to provide ROS to the cell membrane of the cancer cell.

To this end, the second protein constituting the cancer cell death-fusion protein may directly or indirectly bind to the cell membrane of the cancer cell.

To this end, the cancer cell death-fusion protein may be systemically or locally administered to a subject having a cancer disease. Here, the cancer cell death-fusion protein is administered in a protein form, not a DNA vector form encoding the protein.

The step of providing a light to produce ROS by the first protein. is carried out by any one selected from:

a light provided from the outside; or

a light provided from reacting the third protein with a substrate.

Here, the ROS generated by the first protein is provided to a cancer cell membrane, thereby inducing an effect of the cancer cell death.

In one embodiment, the method for inducing a cancer cell death may comprises:

i) preparing a cancer cell death-fusion protein comprising a first protein for generating reactive oxygen species (ROS); and a second protein for specifically binding to a cell membrane of the cancer cell;

ii) inducing the cancer cell death-fusion protein to be attached to the cell membrane of the cancer cell; and

iii) providing a light provided from the outside to produce ROS by the first protein.

In another embodiment, the method for inducing a cancer cell death may comprises:

i) preparing a cancer cell death-fusion protein comprising a first protein for generating reactive oxygen species (ROS); a second protein for specifically binding to a cell membrane of the cancer cell; and a third protein for providing a light;

ii) inducing the cancer cell death-fusion protein to be attached to the cell membrane of the cancer cell; and

iii) providing a light by reacting the third protein with the substrate, after adding a substrate for produce ROS by the first protein.

In another embodiment, the method for inducing a cancer cell death may comprises:

i) preparing a cancer cell death-fusion protein comprising a first protein for generating reactive oxygen species (ROS); a second protein for specifically binding to a cell membrane of the cancer cell; and a third protein for providing a light;

ii) inducing the cancer cell death-fusion protein to be attached to the cell membrane of the cancer cell; and

iii) providing a light provided from the outside to produce ROS by the first protein.

In still another embodiment, the method for inducing a cancer cell death may comprises:

i) preparing a cancer cell death-fusion protein comprising a first protein for generating reactive oxygen species (ROS); and a second protein for specifically binding to a cell membrane of the cancer cell;

ii) inducing the cancer cell death-fusion protein to be attached to the cell membrane of the cancer cell; and

iii) providing a light by reacting the third protein with a substrate for produce ROS by the first protein.

In the above method, the first protein may be an one selected from, for example, KillerRed, MiniSOG, SOPP, FPFB, SuperNova, mKate2, and KillerOrange. In one embodiment, KillerRed or MiniSOG may be used. The ROS generated by the first protein may be superoxide (O₂⁻), hydroxyl radical (HO), singlet oxygen (¹O₂), hydrogen peroxide (H₂O₂), or hypochlorous acid (HOCl), but the present application is not limited thereto. For example, when the first protein is KillerRed, superoxide may be generated. In another example, when the second protein is MiniSOG, singlet oxygen may be generated.

The second protein may be any one selected from, for example, a protein capable of specifically binding to specific receptors expressed on a surface of the cancer cell; a protein capable of specifically binding to membrane protein constituting the cancer cell membrane; a protein capable of specifically binding to a ligand which is able to specifically bind to the specific receptor expressed on the surface of the cancer cell, or a ligand which is able to specifically binding to membrane protein constituting a cancer cell membrane; a protein capable of binding to a specific region of an antibody capable of specifically binding to a protein expressed on the surface of the cancer cell; and a peptide that has permeability to a cell membrane of the cancer cell; and in one embodiment, the second protein may be a WXEAAYQRFL sequence, for example, a WLEAAYQRFL sequence.

The third protein may be any one selected from, for example, Photobacteria luciferase, Firefly luciferase, Railroad worm luciferase, Renilla luciferase, Gaussia luciferase, Metridia luciferase, Cypridiana luciferase, and Oplophorus luciferase (Nanoluc™). In one embodiment, the third protein may use RLuc8 or RLuc8.6.

When the method for inducing a cancer cell death uses a light provided from the outside, it may be easy to kill a cancer cell exposed on a surface of the human body.

When a light provided from the outside is provided, for example, a light such as LED or laser is used, since there is a limitation of irradiated light being provided to the direct surface of a subject, the method for inducing a cancer cell death may be performed by directly irradiating a skin cancer part or a corresponding incision part with light after surgery. Skin cancer and the like may be treated using the above-described method.

When the method for inducing a cancer cell death is a method of reacting a third protein with a substrate, it may be easy to kill cancer cells in an unexposed part, for example, an internal organ.

Since the fusion protein of the present application provides light by itself when the third protein reacts with a substrate, a light may be effectively provided even to a cancer cell deep in cancer tissue and not exposed to the outside. This method may treat various types of a cancer.

### EXAMPLES

Hereinafter, the present application will be described in further detail with reference to examples.

These examples are merely provided to describe the present application in further detail, and it will be apparent to those of ordinary skill in the art that the scope of the present application is not limited by these examples.

Experimental Materials

In examples of the present application,

as a first protein, KillerRed (referred to as KR) and MiniSOG (referred to as MS);

as a second protein, a lead peptide (LP)—the peptide specifically binding to a membrane protein of a specific type of cancer cell, which is represented by SEQ ID NO. 6 (WLEAAYQRFL), known to recognize membrane proteins in the cell membranes of neuroblastoma cell lines such as WAC-2, SH-EP and TET21N and breast cancer cell lines such as MDA-MB-435, MDA-MB-231 and MCF-7 (Zhang, J. B et al, Cancer Lett 171, 153-164 (2001); Ahmed, S et al, Anal Chem 82, 7533-7541 (2010)); and

as a third protein, Renilla luciferase 8.6 (referred to as RLuc8.6) and Renilla luciferase 8 (referred to as RLuc8), were used.

SEQ ID NO. 7: pRSET-KillerRed (31kDa)
MRGSHHHHHHGMASMTGGQQMGRDLYDDDDKDPMLCCMRRTKQVEKNDED
QKISEGGPALFQSDMTFKIFIDGEVNGQKFTIVADGSSKFPHGDFNVHAV
CETGKLPMSWKPICHLIQYGEPFFARYPDGISHFAQECFPEGLSIDRTVR
FENDGTMTSHHTYELDDTCVVSRITVNCDGFQPDGPIMRDQLVDILPNET
HMFPHGPNAVRQLAFIGFTTADGGLMMGHFDSKMTFNGSRAIEIPGPHFV
TIITKQMRDTSDKRDHVCQREVAYAHSVPRITSAIGSDED
SEQ ID NO. 8: pRSET-RLuc8.6-KillerRed (69kDa)
MRGSHHHHHHGMASMTGGQQMGRDLYDDDDKDPMASKVYDPEQRKRMITG
PQWWARCKQMNVLDSFINYYDSEKHAENAVIFLHGNATSSYLWRHVVPHI
EPVARCIIPDLIGMGKSGKSGNGSYRLLDHYKYLTAWFELLNLPKKIIFV
GHDWGSALAFHYAYEHQDRIKAIVHMESVVDVIESWMGWPDIEEELALIK
SEEGEKMVLENNFFVETLLPSKIMRKLEPEEFAAYLEPFKEKGEVRRPTL
SWPREIPLVKGGKPDVVQIVRNYNAYLRASDDLPKLFIESDPGFFYNAIV
EGAKKFPNTEFVKVKGLHFLQEDAPDEMGKYIKSFVERVLKNEQEFGGGM
LCCMRRTKQVEKNDEDQKISEGGPALFQSDMTFKIFIDGEVNGQKFTIVA
DGSSKFPHGDFNVHAVCETGKLPMSWKPICHLIQYGEPFFARYPDGISHF
AQECFPEGLSIDRTVRFENDGTMTSHHTYELDDTCVVSRITVNCDGFQPD
GPIMRDQLVDILPNETHMFPHGPNAVRQLAFIGFTTADGGLMMGHFDSKM
TFNGSRAIEIPGPHFVTIITKQMRDTSDKRDHVCQREVAYAHSVPRITSA
IGSDED
SEQ ID NO. 9: pRSET-RLuc8.6-KillerRed-Lead
peptide (71kDa)
MRGSHHHHHHGMASMTGGQQMGRDLYDDDDKDPMASKVYDPEQRKRMITG
PQWWARCKQMNVLDSFINYYDSEKHAENAVIFLHGNATSSYLWRHVVPHI
EPVARCIIPDLIGMGKSGKSGNGSYRLLDHYKYLTAWFELLNLPKKIIFV
GHDWGSALAFHYAYEHQDRIKAIVHMESVVDVIESWMGWPDIEEELALIK
SEEGEKMVLENNFFVETLLPSKIMRKLEPEEFAAYLEPFKEKGEVRRPTL
SWPREIPLVKGGKPDVVQIVRNYNAYLRASDDLPKLFIESDPGFFYNAIV
EGAKKFPNTEFVKVKGLHFLQEDAPDEMGKYIKSFVERVLKNEQEFGGGM
LCCMRRTKQVEKNDEDQKISEGGPALFQSDMTFKIFIDGEVNGQKFTIVA
DGSSKFPHGDFNVHAVCETGKLPMSWKPICHLIQYGEPFFARYPDGISHF
AQECFPEGLSIDRTVRFENDGTMTSHHTYELDDTCVVSRITVNCDGFQPD
GPIMRDQLVDILPNETHMFPHGPNAVRQLAFIGFTTADGGLMMGHFDSKM
TFNGSRAIEIPGPHFVTIITKQMRDTSDKRDHVCQREVAYAHSVPRITSA
IGSDEDGGGWLEAAYQRFL
SEQ ID NO. 10: pRSET-MiniSOG (13kDa)
MRGSHHHHHHGMASMTGGQQMGRDLYDDDDKDPMEKSFVITDPRLPDNPI
IFASDGFLELTEYSREEILGRNGRFLQGPETDQATVQKIRDAIRDQREIT
VQLINYTKSGKKFWNLLHLQPMRDQKGELQYFIGVQLDG
SEQ ID NO. 11: pRSET-RLuc8-MiniSOG (53kDa)
MRGSHHHHHHGMASMTGGQQMGRDLYDDDDKDPMASKVYDPEQRKRMITG
PQWWARCKQMNVLDSFINYYDSEKHAENAVIFLHGNATSSYLWRHVVPHI
EPVARCIIPDLIGMGKSGKSGNGSYRLLDHYKYLTAWFELLNLPKKIIFV
GHDWGAALAFHYAYEHQDRIKAIVHMESVVDVIESWDEWPDIEEDIALIK
SEEGEKMVLENNFFVETVLPSKIMRKLEPEEFAAYLEPFKEKGEVRRPTL
SWPREIPLVKGGKPDVVQIVRNYNAYLRASDDLPKLFIESDPGFFSNAIV
EGAKKFPNTEFVKVKGLHFLQEDAPDEMGKYIKSFVERVLKNEQEFGGGM
EKSFVITDPRLPDNPIIFASDGFLELTEYSREEILGRNGRFLQGPETDQA
TVQKIRDAIRDQREITVQLINYTKSGKKFWNLEHLQPMRDQKGELQYFIG
VQLDG
SEQ ID NO. 12: pRSET-RLuc8-MiniSOG-Lead
Peptide (54kDa)
MRGSHHHHHHGMASMTGGQQMGRDLYDDDDKDPMASKVYDPEQRKRMITG
PQWWARCKQMNVLDSFINYYDSEKHAENAVIFLHGNATSSYLWRHVVPHI
EPVARCIIPDLIGMGKSGKSGNGSYRELDHYKYLTAWFELLNLPKKIIFV
GHDWGAALAFHYAYEHQDRIKAIVHMESVVDVIESWDEWPDIEEDIALIK
SEEGEKMVLENNFFVETVLPSKIMRKLEPEEFAAYLEPFKEKGEVRRPTL
SWPREIPLVKGGKPDVVQIVRNYNAYLRASDDLPKLFIESDPGFFSNAIV
EGAKKFPNTEFVKVKGEHFLQEDAPDEMGKYIKSFVERVEKNEQEFGGGM
EKSFVITDPRLPDNPIIFASDGFLELTEYSREEILGRNGRFLQGPETDQA
TVQKIRDAIRDQREITVQLINYTKSGKKFWNLEHLQPMRDQKGELQYFIG
VQLDGGGGWLEAAYQRFL

SEQ ID NO. 7: pRSET-KillerRed, SEQ ID NO. 8: pRSET-RLuc8.6-KillerRed, and SEQ ID NO. 9: pRSET-RLuc8.6-KillerRed-Lead peptide were recombined by purchasing a pCS2-NXE+mem-KillerRed plasmid from Addgene (USA). SEQ ID NO. 10: pRSET-MiniSOG, SEQ ID NO. 11: pRSET-RLuc8-MiniSOG, and SEQ ID NO. 12: pRSET-RLuc8-MiniSOG-Lead peptide were recombined by purchasing a mCherry-miniSOG-N1 plasmid from Addgene (USA). Each plasmid was cloned using specific primers (SEQ ID NOs: 13 to 28).

All primers were purchased from Macrogen (Korea). Coelenterazine-h (Co-h), which is a wild-type 2-deoxy derivative, was purchased from Nanolight Technology (USA).

All of the other reagents were commercially available, and reagents with the highest purity grade were purchased and used. Sequencing was analyzed using the sequencing service of Macrogen (Korea).

### Example 1: Expression and Purification of Fusion Proteins

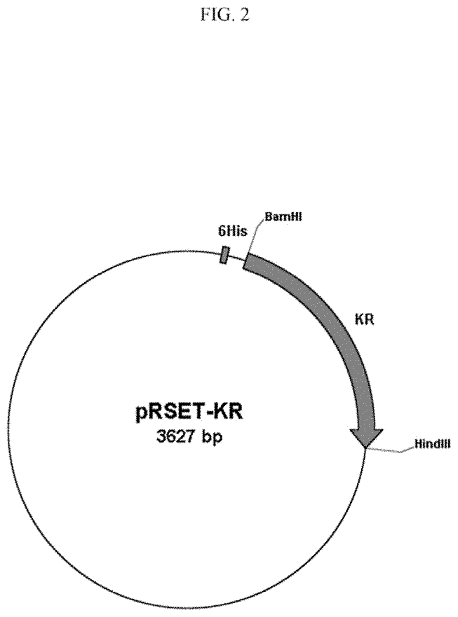

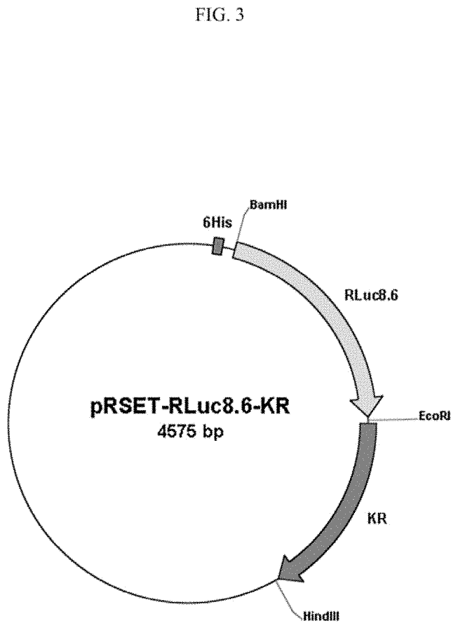

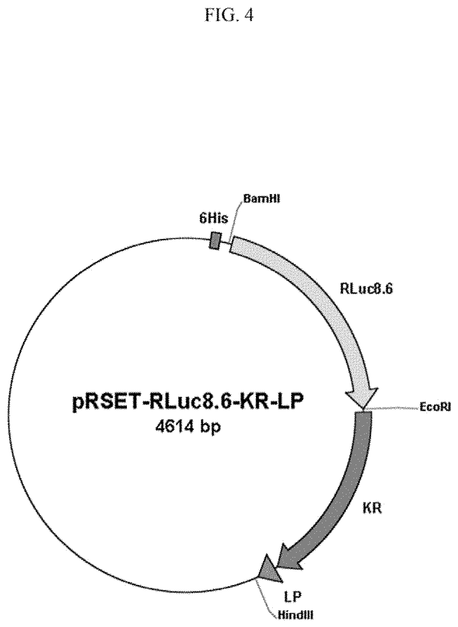

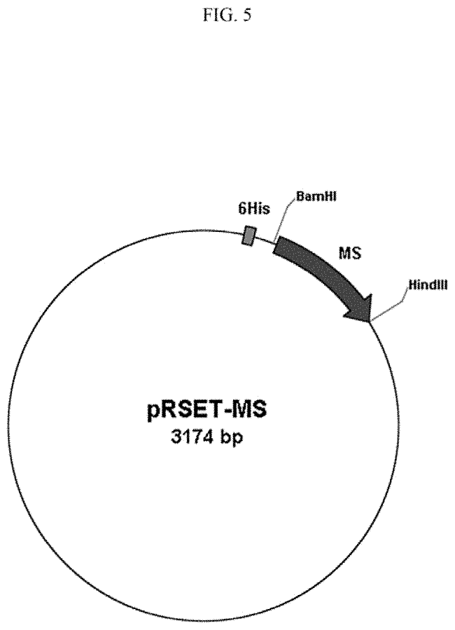

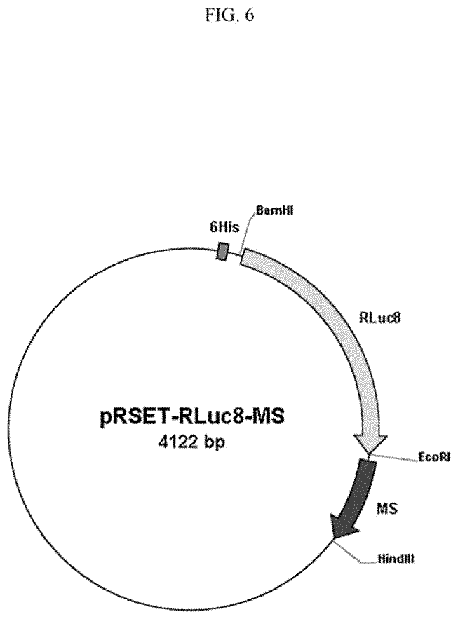

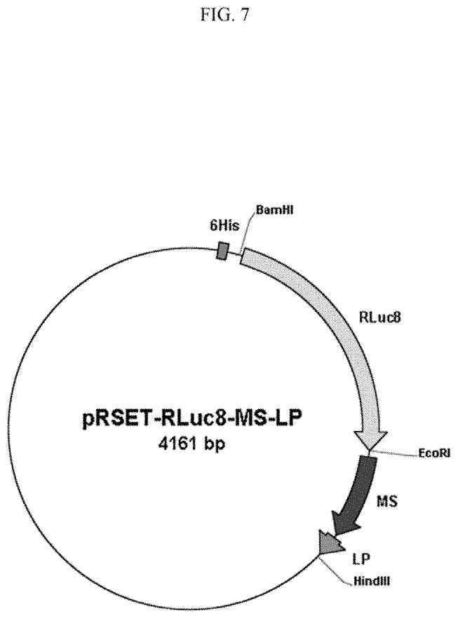

Each of plasmids of FIGS. 2 to 7 was transformed into E. coli strain BL21 cells. The transformed strain (bacteria) was cultured using 500 mL of Luria-Bertani (LB) broth containing 100 μg/mL ampicillin at 37° C. until an optical density (OD) reached 0.9 at 600 nm. Protein expression was induced by adding 1 mM IPTG, and the strain was further cultured at 20° C. for 24 hours. The cells were harvested by centrifugation at 7,800 rpm for 20 minutes.

The obtained cell pellet was resuspended in 20 mL of lysis buffer (lysis buffer; 50 mM NaH₂PO₄, 300 mM NaCl, 10 mM imidazole, pH 8.0) and 2 mg/mL lysozyme, and disrupted by ultrasonication. The disrupted crude cell extract was centrifuged at 14,000 rpm for 20 minutes, the supernatant was filtered, and then incubated with 1 mL of Ni-NTA beads at 4° C. for 24 hours while shaking. A flow-through was removed, the beads were washed with washing buffer (50 mM NaH₂PO₄, 300 mM NaCl and 50 mM imidazole, pH 8.0). The bound protein was eluted with a linear gradient by setting the concentration of imidazole in washing buffer to be 0.5M. A fraction containing the expressed protein was dialyzed, and concentrated with PBS 1×.

Example 1 was carried out to obtain proteins encoded by SEQ ID NOs: 7 to 12, respectively.

### Example 2: Electrophoresis of Proteins

The proteins prepared in Example 1 were separated and identified by molecular weight through sodium dodecylsulfate-polyacrylamide gel electrophoresis (SDS-PAGE).

A gel was prepared using a running gel (3.35 mL of DW, 4 mL of 30% acrylamide, 2.5 mL of 1.5M Tris-HCl (pH 8.8), 100 μL of 10% SDS, 50 μL of 10% APS, 10 μL of TEMED) and a stacking gel (1.5 mL of DW, 330 μL of 30% acrylamide, 630 μL of 1M Tris-HCl (pH 6.8), 25 μL of 10% SDS, 12.5 μL of 10% APS, 5 μL of TEMED), and the proteins prepared in Example 1 was loaded into wells, and electrophoresis was performed at 100V for 100 minutes.

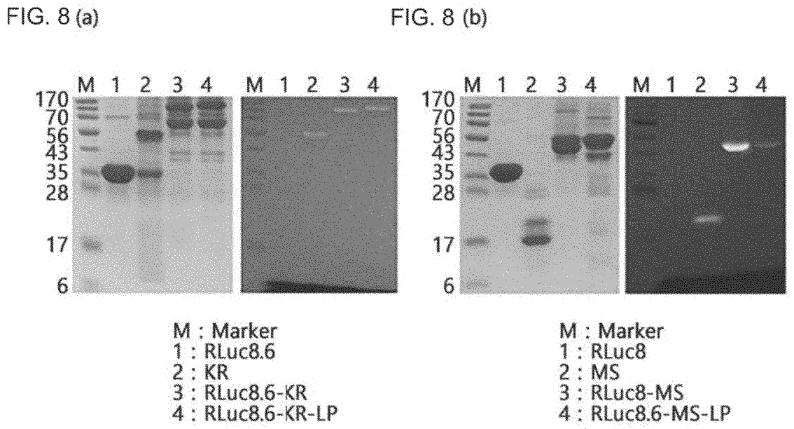

The electrophoresis result for the proteins is illustrated in FIG. 8.

As a result,

it was seen that RLuc8.6 is 38 kDa, KillerRed is 31 kDa, RLuc8.6-KillerRed is 69 kDa, and RLuc8.6-KillerRed-Lead peptide is 71 kDa (FIG. 8(a)), and

it was seen that RLuc8 is 38 kDa, MiniSOG is 13 kDa, RLuc8-MiniSOG is 53 kDa, and RLuc8-MiniSOG-Lead peptide is 54 kDa (FIG. 8(b)).

### Example 3: Fluorescence and Bioluminescence Assays for Proteins

100 μL of buffer (1×PBS) containing the protein purified in Example 1 (final concentration: 10 μM) was dispensed into a 96-well plate (SPL Cell Culture Plate, 96 well (SPL, Cat #32096)). Afterward, a fluorescence signal level was detected by fluorescence assay using a Thermo Scientific™ Varioskan™ Flash Multimode Reader.

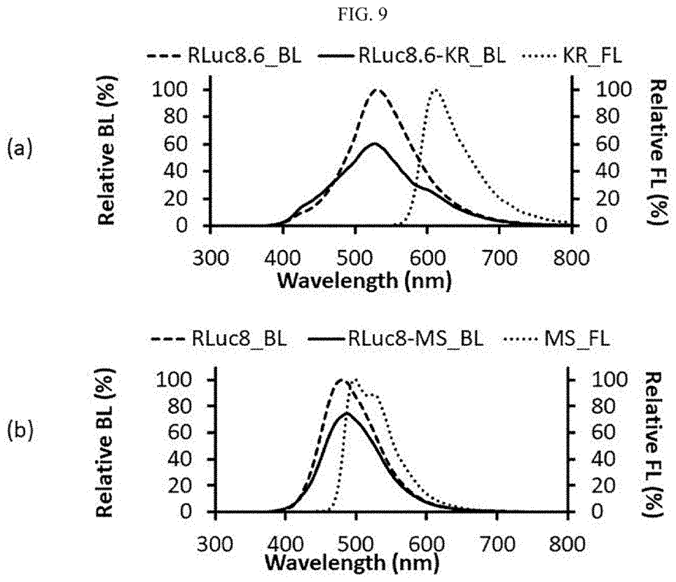

Referring to FIG. 9, it can be confirmed that the excitation wavelength of the KillerRed was 585 nm, and the emission wavelength thereof was 610 nm (FIG. 9(a)).

In addition, it can be confirmed that the excitation wavelength of the MiniSOG was 448 nm, and the emission wavelength thereof was 500 nm and 528 nm (FIG. 9(b)).

In addition, 100 μL of buffer (1×PBS) containing the protein purified in Example 1 (final concentration: 1 μM) was dispensed into a 96-well plate (SPL Cell Culture Plate, 96 well (SPL, Cat #30196)), and bioluminescence assay was then performed using 50 μL of buffer (1×PBS) containing a Co-h substrate solution (final concentration: 50 μM), which is a wild-type 2-deoxy derivative. Bioluminescence intensity was immediately measured at a wavelength of 300 to 800 nm using a Thermo Scientific™ Varioskan™ Flash Multimode Reader.

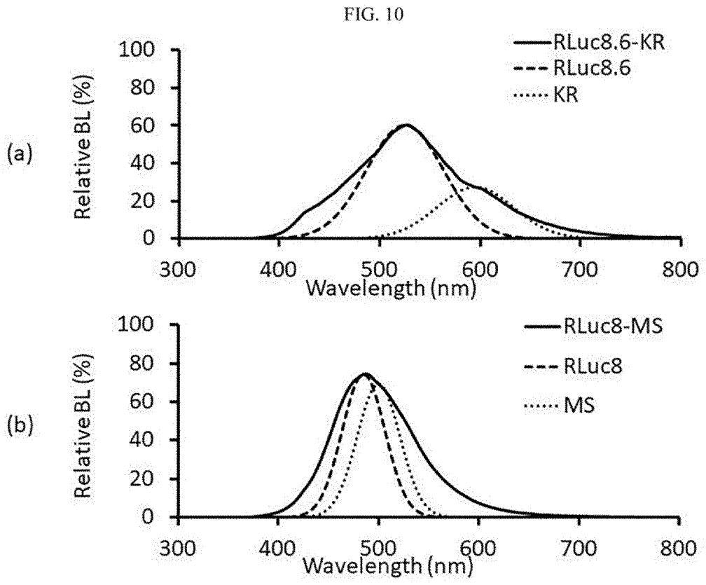

Referring to FIG. 10, it can be confirmed that bioluminescence resonance energy transfer (BRET) occurred by the emission of RLuc8.6 at 535 nm and the emission of KillerRed at 610 nm (FIG. 10(a)).

In addition, it can be confirmed that BRET occurred by the emission of RLuc8 at 480 nm and the emission of MiniSOG at 500 nm (FIG. 10(b)).

### Example 4: Effect of Generating ROS According to Concentration of Coelenterazine-h Substrate

A ROS-generating effect of the RLuc8.6-KR protein was confirmed using 100 μL of buffer (50 mM HEPES-KOH, 24° C., pH 7.4) containing RLuc8.6-KR protein (final concentration: 10 μM), dihydroethidium (DHE, Sigma Aldrich, USA; final concentration of 100 μM) whose fluorescence intensity decreases in the presence of superoxide and a Co-h substrate solution. The rate of decrease in fluorescence intensity was measured at the excitation wavelength of 370 nm and the emission wavelength of 420 nm of DHE using a plate reader.

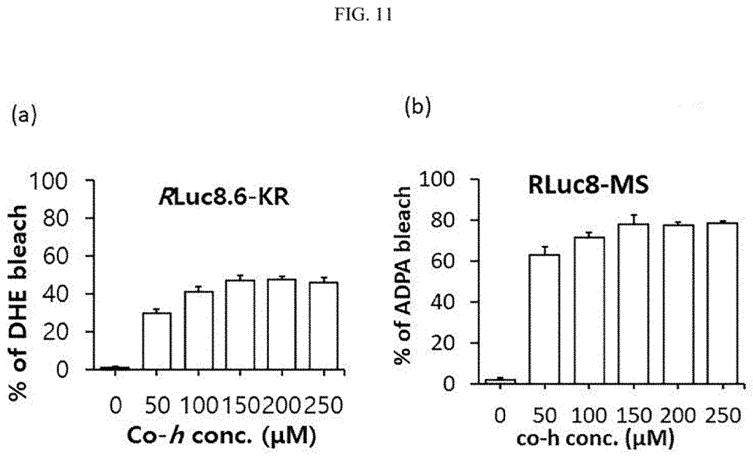

Referring to FIG. 11(a), it can be confirmed that the generation rate of the superoxide confirmed with dihydroethidium (DHE) was increased until the final concentration of the Co-h substrate solution became 150 μM, and then was constantly maintained.

A ROS-generating effect of the RLuc8-MS protein was confirmed using 100 μL of buffer (50 mM HEPES-KOH, 24° C., pH 7.4) containing RLuc8-MS protein (final concentration: 10 μM), anthracene-9,10-dipropionic acid (ADPA, Abcam., UK; final concentration: 50 μM) whose fluorescence intensity decreases in the presence of singlet oxygen, flavin mononucleotide (FMN) (final concentration: 150 μM) and a Co-h substrate solution. The rate of decrease in fluorescence intensity was measured at the excitation wavelength of 380 nm and the emission wavelength of 430 nm of ADPA using a plate reader.

Referring to FIG. 11(b), it can be confirmed that the generation rate of the singlet oxygen confirmed with ADPA was increased until the concentration of the Co-h substrate solution became 150 μM and then was constantly maintained.

### Example 5: Measurement of ROS Generation Rate According to Reaction Time with h-Co Substrate

A ROS-generating effect of RLuc8.6-KR protein was measured using RLuc8.6-KR protein (final concentration: 10 μM) and DHE (final concentration: 100 μM) whose fluorescence intensity decreases in the presence of superoxide, and

a ROS-generating effect of the RLuc8-MS protein was measured using RLuc8-MS (final concentration: 10 μM) and ADPA (final concentration: 50 μM) whose fluorescence intensity decreases in the presence of singlet oxygen.

Here, the active oxygen generation rate of each protein was measured using 100 μL of buffer (50 mM HEPES-KOH, 24° C., pH 7.4) containing FMN (final concentration: 150 μM) and a Co-h substrate solution (final concentration: 150 μM).

The rate of decrease in fluorescence intensity was measured at the excitation wavelength of 370 nm and the emission wavelength of 420 nm of DHE using a plate reader. In addition, the rate of decrease in fluorescence intensity was measured at the excitation wavelength of 380 nm and the emission wavelength of 430 nm of ADPA using a plate reader.

Referring to FIG. (a), it can be confirmed that the generation rate of superoxide was increased until the reaction time with the Co-h substrate became 30 minutes, and then was constantly maintained.

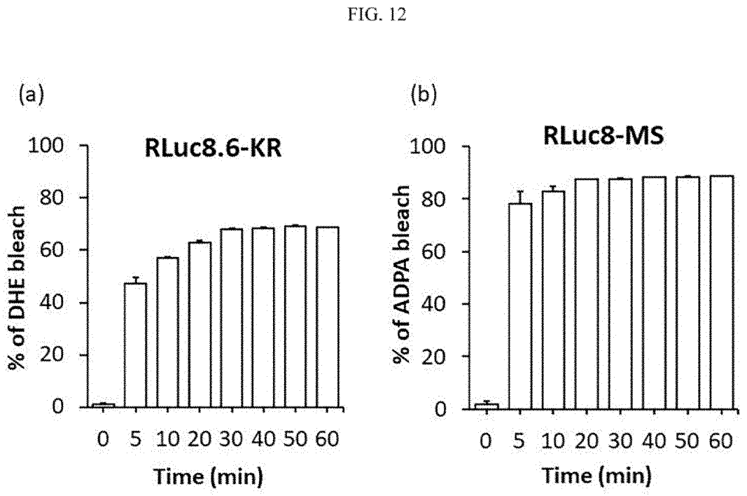

Referring to FIG. 12(b), it can be confirmed that the generation rate of singlet oxygen was increased until the reaction time with the Co-h substrate became 20 minutes, and then was constantly maintained.

### Example 6: Measurement of Reactive Oxygen Species Generation Rate of Protein after Light Irradiation

A ROS generation rate was measured using 100 μL of buffer (50 mM HEPES-KOH, 24° C., pH 7.4) containing the KR protein, RLuc8.6-KR protein (final concentration: 10 μM) and DHE (final concentration: 100 μM) whose fluorescence intensity decreases in the presence of superoxide.

A ROS generation rate was measured using 100 μL of buffer (50 mM HEPES-KOH, 24° C., pH 7.4) containing MS, RLuc8-MS (final concentration: 10 μM), DHE (final concentration: 100 μM) whose fluorescence intensity decreases in the presence of superoxide and FMN (final concentration: 150 μM).

After irradiation with light (referred to as +LED Light) at 10 mW/cm² for 30 minutes, the rate of decrease in fluorescence intensity was measured at the excitation wavelength of 370 nm and the emission wavelength of 420 nm of DHE using a plate reader.

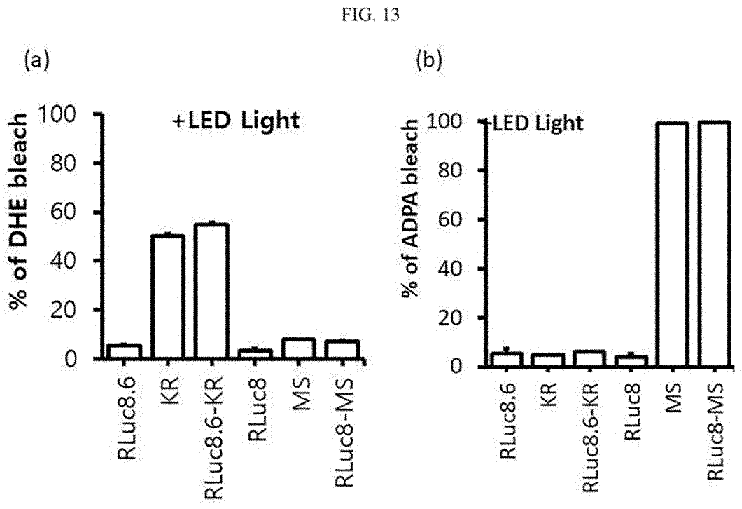

Referring to FIG. 13(a), it can be confirmed that KillerRed had a higher superoxide generation rate, which is confirmed with DHE, than MiniSOG.

A ROS generation rate was measured using 100 μL of buffer (50 mM HEPES-KOH, 24° C., pH 7.4) containing the KR protein, RLuc8.6-KR protein (final concentration: 10 μM) and ADPA (final concentration: 50 μM) whose fluorescence intensity decreases in the presence of singlet oxygen.

A ROS generation rate was measured using 100 μL of buffer (50 mM HEPES-KOH, 24° C., pH 7.4) containing MS, RLuc8-MS (final concentration: 10 μM), ADPA (final concentration: 50 μM) whose fluorescence intensity decreases in the presence of singlet oxygen and FMN (final concentration: 150 μM).

After irradiation with light at 10 mW/cm² for 30 minutes, the rate of decrease in fluorescence intensity was measured at the excitation wavelength of 380 nm and the emission wavelength of 430 nm of ADPA using a plate reader.

Referring to FIG. 13(b), it can be confirmed that MiniSOG had a higher singlet oxygen generation rate, which is confirmed with ADPA, than KillerRed.

### Example 7: Measurement of ROS Generation Rate of Protein after Co-h Substrate Reaction

A ROS generation rate was measured using 100 μL of buffer (50 mM HEPES-KOH, 24° C., pH 7.4) containing the KR protein, RLuc8.6-KR protein (final concentration: 10 μM), DHE (final concentration: 100 μM) whose fluorescence intensity decreases in the presence of superoxide and a Co-h substrate solution (final concentration: 150 μM).

A ROS generation rate was measured using 100 μL of buffer (50 mM HEPES-KOH, 24° C., pH 7.4) containing MS, RLuc8-MS (final concentration: 10 μM), DHE (final concentration: 100 μM) whose fluorescence intensity decreases in the presence of superoxide, FMN (final concentration: 150 μM) and a Co-h substrate solution (final concentration: 150 μM). The rate of decreasing fluorescence intensity was measured at an excitation wavelength of 370 nm and an emission wavelength of 420 nm of DHE using a plate reader.

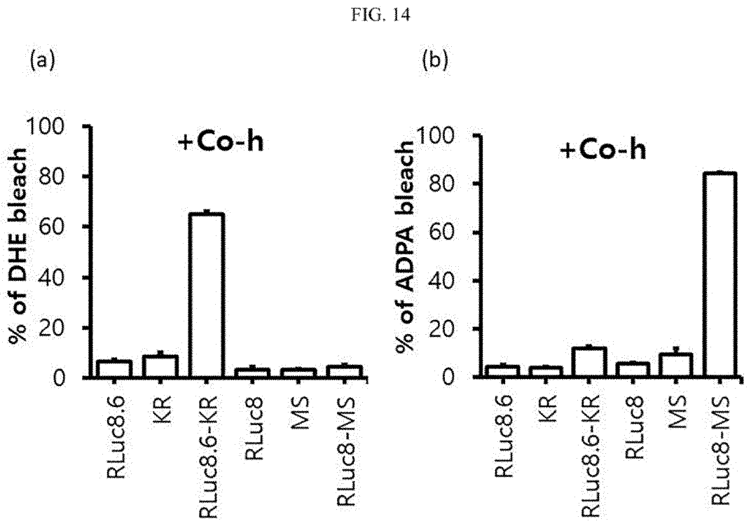

Referring to FIG. 14(a), it can be confirmed that RLuc8.6-KR had the highest superoxide generation rate, which is confirmed with DHE.

A ROS generation rate was measured using 100 μL of buffer (50 mM HEPES-KOH, 24° C., pH 7.4) containing the KR protein, RLuc8.6-KR protein (final concentration: 10 μM) and ADPA (final concentration: 50 μM) whose fluorescence intensity decreases in the presence of singlet oxygen and a Co-h substrate solution (final concentration: 150 μM).

A ROS generation rate was measured using 100 μL of buffer (50 mM HEPES-KOH, 24° C., pH 7.4) containing MS, RLuc8-MS (final concentration: 10 μM), ADPA (final concentration: 50 μM) whose fluorescence intensity decreases in the presence of singlet oxygen, FMN (final concentration: 150 μM) and a Co-h substrate solution (final concentration: 150 μM).

The rate of decreasing fluorescence intensity was measured at the excitation wavelength of 380 nm and the emission wavelength of 430 nm of ADPA using a plate reader.

Referring to FIG. 14(b), it can be confirmed that RLuc8-MS had the highest singlet oxygen generation rate, which is confirmed with ADPA.

### Example 8: Measurement of Reactive Oxygen Species Generation Rate of Protein by Co-h Substrate Reaction after ROS Scavenger Treatment

A ROS generation rate was measured by reaction of 100 μL of buffer (50 mM HEPES-KOH, 24° C., pH 7.4) containing RLuc8.6-KR protein (final concentration: 10 μM), DHE (final concentration: 100 μM) whose fluorescence intensity decreases in the presence of superoxide, ROS scavenger (SOD (superoxide scavenger, final concentration 800U/ml), sodium azide (Singlet oxygen scavenger; final concentration: 100 mM) and D-mannitol (hydroxyl radical scavenger; final concentration: 100 mM), respectively) for 30 minutes.

For the ROS generation of a protein by a light, 2.6 μL of a Co-h substrate solution was added [Co-h (2.5 mg/mL, 6,000 μM), final concentration: 150 μM, Total: 102.6 μl] and then reacted at room temperature for 30 minutes. The rate of decreasing fluorescence intensity was measured at an excitation wavelength of 370 nm and an emission wavelength of 420 nm of DHE using a plate reader.

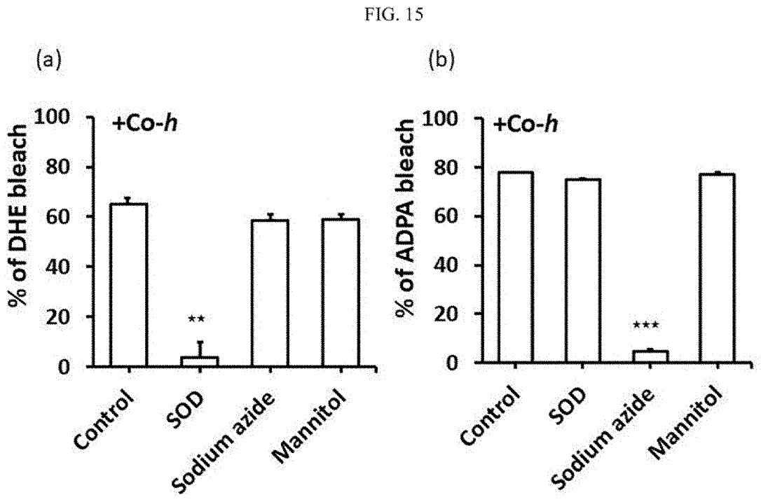

Referring to FIG. 15(a), the superoxide generation rate of KillerRed confirmed with DHE was lowest when SOD was treated, confirming that superoxide generation is inhibited by SOD.

A ROS generation rate was measured by reaction of 100 μL of buffer (50 mM HEPES-KOH, 24° C., pH 7.4) containing RLuc8-MS (final concentration: 10 μM), ADPA (final concentration: 50 μM) whose fluorescence intensity decreases in the presence of singlet oxygen, FMN (final concentration: 150 μM), ROS scavenger SOD (superoxide scavenger, final concentration: 800 U/ml), sodium azide (singlet oxygen scavenger, final concentration: 100 mM) and D-mannitol (hydroxyl radical scavenger, final concentration: 100 mM) for 30 minutes. For the ROS generation of a protein by light, 2.6 μL of a Co-h substrate solution was added [Co-h (2.5 mg/mL, 6000 μM), final concentration: 150 μM, total: 102.6 μl] and reacted at room temperature for 30 minutes. The rate of decreasing fluorescence intensity was measured at the excitation wavelength of 380 nm and the emission wavelength of 430 of ADPA nm using a plate reader.

Referring to FIG. 15(b), the singlet oxygen generation rate of MiniSOG confirmed with ADPA was lowest when sodium azide was treated, confirming that singlet oxygen generation was inhibited by sodium azide.

### Example 9: Measurement of Stability of Bioluminescence Intensity after Co-h Substrate Reaction

Bioluminescence assay was performed by reaction of 30 μL of buffer (1×PBS) containing the purified protein RLuc8.6 or RLuc8.6-KR-LP protein, or RLuc8 or RLuc8-MS-LP protein (final concentration: 10 μM) and 0.8 μL of a Co-h substrate solution (final concentration: 150 μM) in an incubator (37° C.). 30 μL of normal mouse serum (Jackson ImmunoResearch, USA) containing the purified protein (final concentration: 10 μM) and 0.8 μL of a Co-h substrate solution (final concentration: 150 μM) were reacted in an incubator (37° C.). Bioluminescence intensity was immediately measured using GLOMAX.

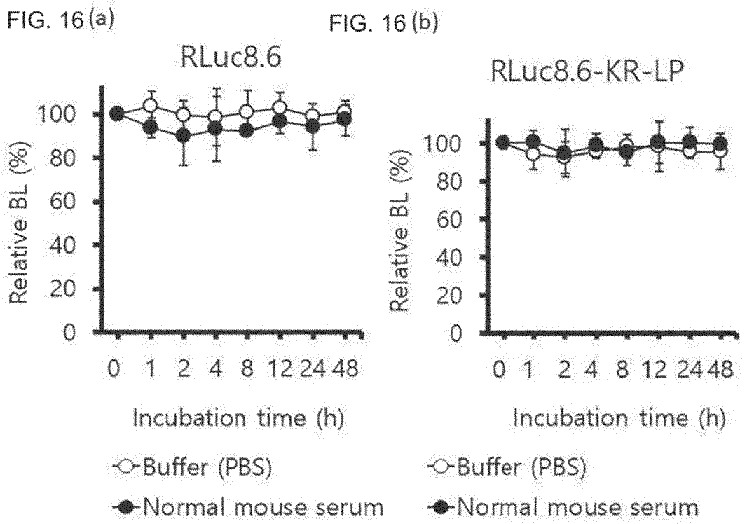

Referring to FIGS. 16(a) and 16(b), it can be confirmed that the luminescence intensity of RLuc8.6 and RLuc8.6-KR-LP proteins in a buffer (1×PBS) or normal mouse serum was constant even when the reaction time during incubation (37° C.) was increased.

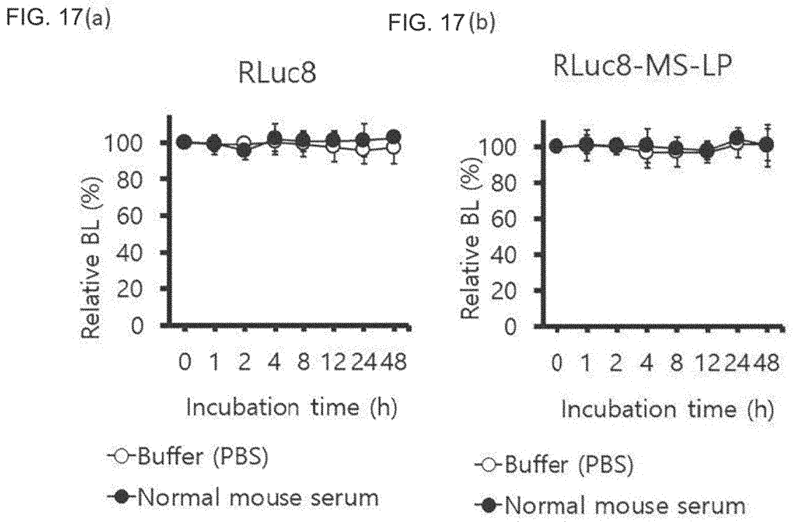

Referring to FIGS. 17(a) and 17(b), it can be confirmed that the luminescence intensity of RLuc8 and RLuc8-MS-LP proteins in a buffer (1×PBS) or normal mouse serum was constant even when the reaction time during incubation (37° C.) was increased.

### Example 10: Measurement of Colorimetric Change of MTT and Cell Viability According to Protein Type Treated to Cells and Light Irradiation Time

MCF-7 cells were seeded at 5×10⁵ cells/mL in a 96-well plate (SPL Cell Culture Plate, 96 well (SPL, Cat #30096)), and cultured at 37° C. for 24 hours (overnight). After the overnight culture, the cells were washed with FBS & Phenol red-free media (RPMI, Well Gene., Korea, Cat #LM011-02), and 100 μL of an FBS-free medium containing the purified protein (final concentration: 10 μM) was added, followed by reaction at 37° C. for 24 hours. After the reaction for 24 hours, the cells were washed with FBS & Phenol red-free media (RPMI), and then 100 μL of FBS & Phenol red-free media (RPMI) was added again. After light irradiation at 10 mW/cm² over time, the cells were washed with FBS & Phenol red-free media (RPMI), and 100 μL of FBS & Phenol red-free media (RPMI) was added again. 10 μL of 3-(4,5-dimethylthiazol-2-yl)-2,5-diphenyltetrazolium bromide (MTT; Cell Biolabs Inc., USA; final concentration: 1×) was added, followed by reaction at 37° C. for 4 hours. 100 μL of a supernatant was removed, and 100 μL of DMSO (Sigma-Aldrich., USA) was added, followed by reaction for 10 minutes. The colorimetric change of MTT according to a protein type treated to the cells and light irradiation time was confirmed, and then cell viability was confirmed by measuring the absorbance intensity at 570 nm using a plate reader.

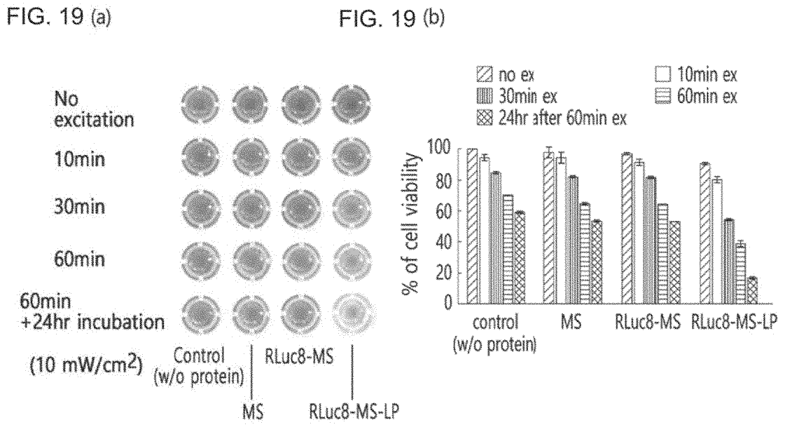

Referring to FIGS. 18 and 19, since the cell treated with KR, RLuc8.6-KR, MS and RLuc8-MS have no a second protein, the cell are alive, and thus it can be seen that MTT enters the mitochondria, and thus the cells are stained pink. However, since the cell treated with RLuc8.6-KR-LP or RLuc8-MS-LP are killed as the reaction time with the Co-h solution increases, it can be seen that the cell are not stained pink.

That is, in the cell treated with RLuc8.6-KR-LP, as the light irradiation time increased, it can be confirmed that the colorimetric change of MTT was increased, and cell viability decreased.

In this example, for a cancer cell, previously expressed KR, RLuc8.6-KR, MS or RLuc8-MS protein is used, and these proteins are not introduced into an interior of the cancer cell due to their sizes. For this reason, to kill the cancer cell using ROS, there is a need for satisfying the requirement of providing ROS in as close to of the cancer cell.

From the experimental result, it can be expected that a second protein, a lead peptide, serves to place the KR, RLuc8.6-KR, MS and RLuc8-MS proteins generating ROS as close as possible to the cancer cell to kill the cancer cell. That is, it is considered that the lead peptide places the proteins as close to of the cancer cell membrane to provide ROS to the cancer cell membrane, thereby inducing the cancer cell death.

### Example 11: Measurement of Colorimetric Change of MTT and Cell Viability According to Protein Type Treated to Cells and Reaction Time with Co-h Substrate

MCF-7 cells were seeded at 5×10⁵ cells/mL in a 96-well plate (SPL Cell Culture Plate, 96 well (SPL, Cat #30096)), and cultured at 37° C. for 24 hours (overnight).

After the culture for 24 hours, the cells were washed with FBS & Phenol red-free media (RPMI), and 100 μL of an FBS-free medium containing the purified protein (final concentration: 10 μM) was added, followed by reaction at 37° C. for 24 hours.

After the reaction for 24 hours, the cells were washed with FBS & Phenol red-free media (RPMI), and then 100 μL of FBS & Phenol red-free media (RPMI) containing a Co-h substrate solution (final concentration: 150 μM) was added. The cells were washed with FBS & Phenol red-free media (RPMI), and 100 μL of fresh FBS & Phenol red-free media (RPMI) were then added.

10 μL of 3-(4,5-dimethylthiazol-2-yl)-2,5-diphenyltetrazolium bromide (MTT) (final concentration: 1×) was added, followed by reaction at 37° C. for 4 hours. After 100 μL of a supernatant was removed, 100 μL of DMSO was added, followed by reaction for 10 minutes. The colorimetric change of MTT according to a protein type treated to the cells and the light irradiation time was checked, and then cell viability was confirmed by measuring the absorbance intensity at 570 nm using a plate reader.

MTT is an assay for measuring cell proliferation or live cells through the presence of blue-violet insoluble 3-(4,5-dimethylthiazol-2-yl)-2,5-diphenyltetrazolium bromide (MTT formazan) in the mitochondria of the live cells, where it has been reduced from a yellow soluble substrate, MTT tetrazolium, by the action of a dehydrogenase.

Referring to FIGS. 20 to 23, it can be seen that, since there is no a second protein, a lead peptide, which has a role of placing the KR, RLuc8.6-KR, MS and RLuc8-MS proteins generating active enzyme as close as possible to cancer cells, MTT enters the mitochondria of live cells and the staining becomes pink. However, since the cells treated with RLuc8.6-KR-LP or RLuc8-MS-LP die as the reaction time with a Co-h solution increases, it can be seen that the cells are not stained pink.

That is, in the cells treated with RLuc8.6-KR-LP or RLuc8-MS-LP, it can be confirmed that the colorimetric change of MTT is increased as reaction time with a Co-h solution is increased, and cell viability is reduced.

### Example 12: Fluorescence Synthesis Images According to Protein Type Treated to Cells and i) Light Irradiation Time or ii) Reaction Time with Co-h Substrate and Measurement of Relative Fluorescence Intensity of SYTOX Green or EthD-1

MCF-7 cells were seeded at 5×10⁵ cells/mL in a 96-well plate (SPL Cell Culture Plate, 96 well (SPL, Cat #30096)), and cultured at 37° C. for 24 hours (overnight). After the culture for 24 hours, the cells were washed with FBS & Phenol red-free media (RPMI), and 100 μL of an FBS-free medium containing the purified protein (final concentration: 10 μM) was added, followed by reaction at 37° C. for 24 hours.

After the reaction for 24 hours, the cells were washed with FBS & Phenol red-free media (RPMI), and then 100 μL of FBS & Phenol red-free media (RPMI) was then added.

After the reaction, 8.1 μL of FMN (final concentration: 150 μM) was added. After reaction for 18 hours or 24 hours, the cells were washed with FBS & Phenol red-free media (RPMI), and 100 μL of FBS & Phenol red-free media (RPMI) containing FMN (final concentration: 150 μM) was added, followed by reaction at 37° C. for 1 hour.

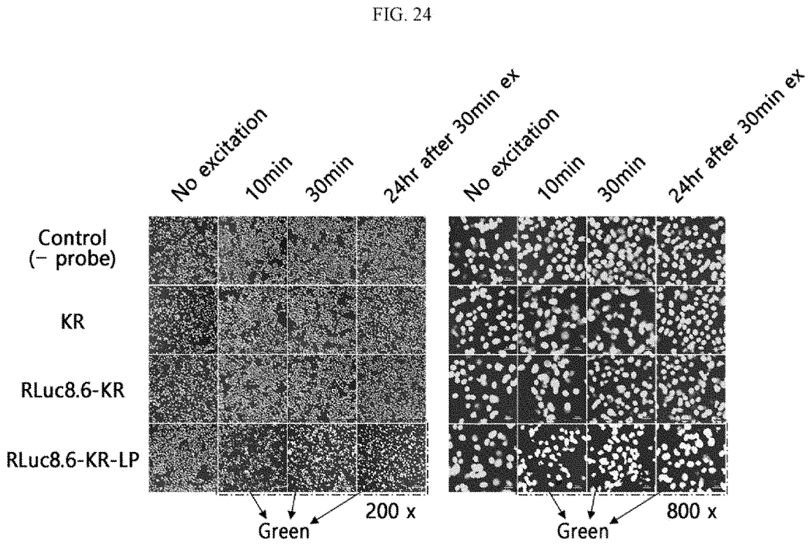

i) Experiment According to Light Irradiation Time (FIGS. 24, 25, 28 and 29)

After the irradiation with light at 10 mW/cm² over time, 50 μL of SYTOX Green (Thermo Scientific Inc., USA; final concentration: 261 nM) staining DNA of dead cells was added and reacted at 37° C. for 30 minutes, and 10 μL of DAPI (Vecta Labs., Australia) staining DNA of live cells was added and reacted for 5 minutes.

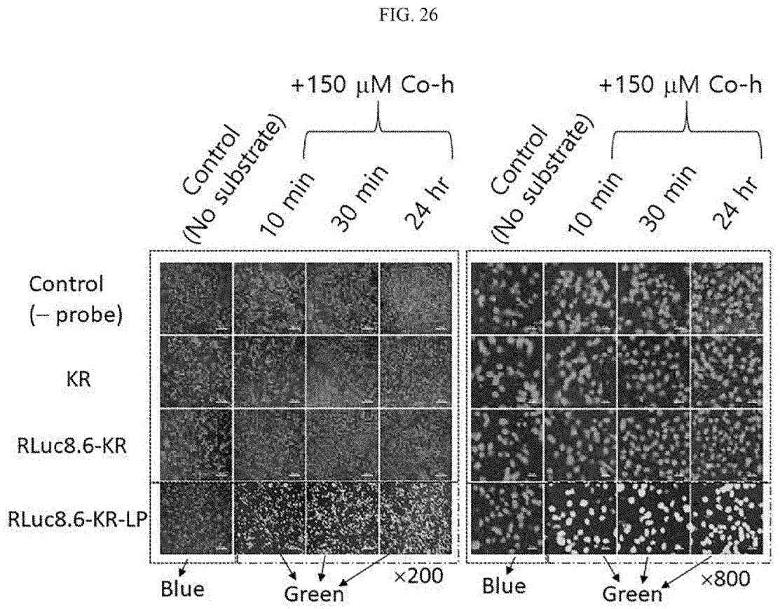

ii) Experiment According to Time to React with Co-h Substrate (FIGS. 26, 27, 30 and 31)

After reaction of 2.6 μL of a Co-h substrate solution (final concentration: 150 μM) over time, 50 μL of SYTOX Green (final concentration: 261 nM) or 90 μL of an Ethidium homodimer (EthD-1; final concentration: 1×), which stains DNA of dead cells, was added and reacted at 37° C. for 30 minutes, and then 10 μL of DAPI staining DNA of live cells was added and reacted for 5 minutes.

SYTOX Green or EthD-1 and DAPI fluorescence images were observed using a confocal microscope according to a protein type treated to cells and i) light irradiation time or ii) reaction time with a Co-h substrate, and cell death was confirmed by measuring relative fluorescence intensity at an excitation wavelength of 504 nm and an emission wavelength of 523 nm of SYTOX Green, and an excitation wavelength of 525 nm and an emission wavelength of 590 nm of EthD-1 using a plate reader.

Whether or not Cell death was observed by fluorescence microscopy using SYTOX Green (dead cell-specific dye, green) or EthD-1 (dead cell-specific dye, red) and DAPI (live cell-specific dye, blue).

Referring to FIGS. 24 to 31, it can be seen that, since the cells are treated with KR, RLuc8.6-KR, MS and RLuc8-MS having no a second protein, a lead peptide, serving to place proteins generating ROS as close as possible to cancer cells, the cells are stained with DAPI even when light is irradiated or reacted with a substrate. However, it can be seen that the cells treated with RLuc8.6-KR-LP or RLuc8-MS-LP are stained with SYTOX Green or EthD-1 as light is irradiated or the reaction time with a substrate (Co-h) solution is increased.

That is, in the case of the RLuc8.6-KR-LP-treated cells, it can be confirmed that, as the light irradiation time or the reaction time with a substrate increases, the number of dead cells increases, and the fluorescence intensity of SYTOX Green or EthD-1 increases.

### Example 13: Confirmation of Cell Death According to Incubation Time after Treatment with Various Co-h Substrate Concentrations Treated to RLuc8.6-KR-LP Protein

MCF-7 cells were seeded at 5×10⁵ cells/mL in a 96-well plate (SPL Cell Culture Plate, 96 well (SPL, Cat #30096)), and cultured at 37° C. for 24 hours (overnight). After the culture for 24 hours, the cells were washed with FBS & Phenol red-free media (RPMI, Well Gene., Korea, Cat #LM011-02), and 100 μL of an FBS-free medium containing the purified protein (final concentration: 10 μM) was added, followed by reaction at 37° C. for 24 hours.

After the reaction for 24 hours, the cells were washed with FBS & Phenol red-free media (RPMI), and then 100 μL of FBS & Phenol red-free media (RPMI) was added again. The reaction was performed by each concentration of the Co-h substrate solution, and then the cells were incubated at 37° C.

After incubation, 50 μL of SYTOX Green (final concentration: 261 nM) staining DNA of dead cells and DAPI 10 μL of DAPI staining DNA of live cells were added, followed by reaction for 5 minutes. Cell death was confirmed by detecting KillerRed, SYTOX Green and DAPI fluorescence according to the light irradiation time for cells using a confocal microscope.

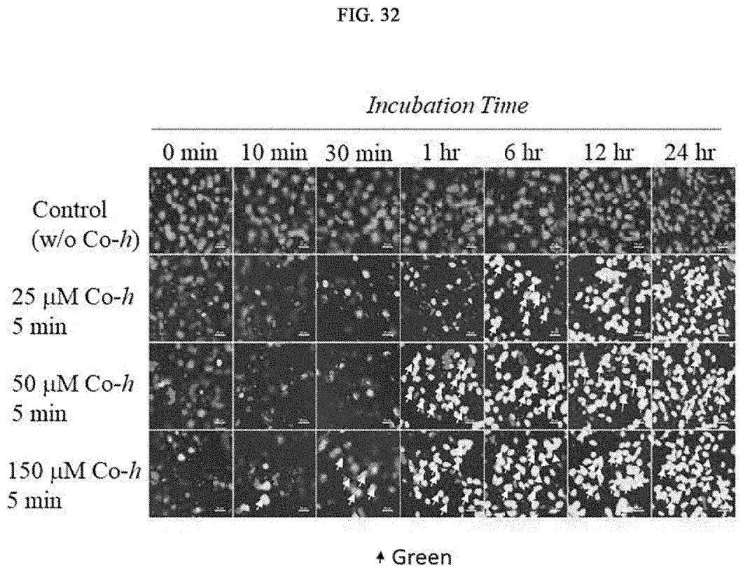

Referring to FIG. 32, it can be confirmed that the number of cells stained with SYTOX Green increased when a cell incubation time of approximately 30 minutes or more had passed after the cells treated with RLuc8.6-KR-LP protein were treated with a 25 μM or 50 μM substrate solution for 5 minutes. However, the cell death according to cell incubation time after a 150 μM substrate solution was treated for 5 minutes can also be confirmed by observing cells stained with SYTOX Green even approximately 10 minutes after cell culture.

Accordingly, the cell death effect according to incubation time after treatment with various concentrations of Co-h substrate solution was demonstrated by observing that, as the concentration of the substrate solution increases, the number of cells stained with SYTOX Green increases within a short time after incubation.

### Example 14: Confirmation of Cell Death According to Incubation Time Per Light Irradiation Time of RLuc8.6-KR-LP Protein-Treated Cells

MCF-7 cells were seeded at 5×10⁵ cells/mL in a 96-well plate (SPL Cell Culture Plate, 96 well (SPL, Cat #30096)), and cultured at 37° C. for 24 hours (overnight). After the culture for 24 hours, the cells were washed with FBS & Phenol red-free media (RPMI, Well Gene., Korea, Cat #LM011-02), and 100 μL of an FBS-free medium containing the purified protein (final concentration: 10 μM) was added, followed by reaction at 37° C. for 24 hours.

After the reaction for 24 hours, the cells were washed with FBS & Phenol red-free media (RPMI), and then 100 μL of FBS & Phenol red-free media (RPMI) was added again. After light irradiation at 10 mW/cm² over time, the cells were incubated at 37° C.

After incubation, 50 μL of SYTOX Green (final concentration: 261 nM) staining DNA of dead cells and 10 μL of DAPI (Vector Laboratories Inc., USA) staining DNA of live cells were added, followed by reaction for 5 minutes. Cell death was confirmed by detecting KillerRed, SYTOX Green and DAPI fluorescence according to the light irradiation time for cells using a confocal microscope.

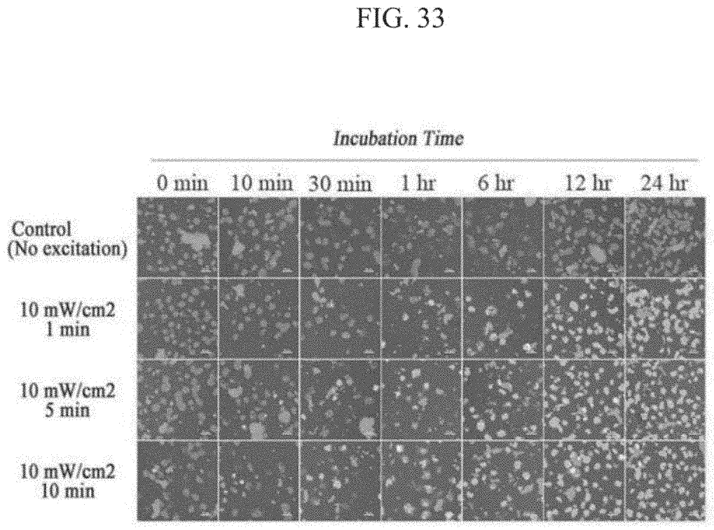

Referring to FIG. 33, it can be confirmed that the number of cells stained with SYTOX Green increased when a cell incubation time of over 1 hour had passed when the RLuc8.6-KR-LP protein-treated cells were irradiated with light at 10 mW/cm² for 1 minute. However, when the cells were irradiated with light at 10 mW/cm² for 5 minutes or 10 minutes, it can be confirmed that from approximately 30 minutes after cell incubation, the number of cells stained with SYTOX Green increased.

Accordingly, it can be demonstrated that the longer the cells were irradiated with light at 10 mW/cm², the higher the cell death rate over cell incubation time.

### Example 15: Confirmation of Cell Death According to the Presence of Serum and Time to Treat RLuc8.6-KR-LP Protein Using Co-h Substrate

MCF-7 cells were seeded at 5×10⁵ cells/mL in a 96-well plate (SPL Cell Culture Plate, 96 well (SPL, Cat #30096)), and cultured at 37° C. for 24 hours (overnight). After the culture for 24 hours, the cells were washed with FBS & Phenol red-free media (RPMI, Well Gene., Korea, Cat #LM011-02), and 100 μL of an FBS-free medium or FBS media (RPMI), containing purified protein (final concentration: 10 μM), was added, followed by reaction at 37° C. over time.

After the reaction, the cells were washed with FBS & Phenol red-free media (RPMI), 100 μL of FBS & Phenol red-free media (RPMI) containing a Co-h substrate solution (final concentration: 150 μM) and SYTOX Green (final concentration: 261 nM) staining DNA of dead cells was added, followed by reaction at 37° C. for 30 minutes.

After the reaction, 10 μL of DAPI staining DNA of live cells was added, followed by reaction for 5 minutes. Cell death was confirmed by observing KillerRed, SYTOX Green and DAPI fluorescence using a confocal microscope.

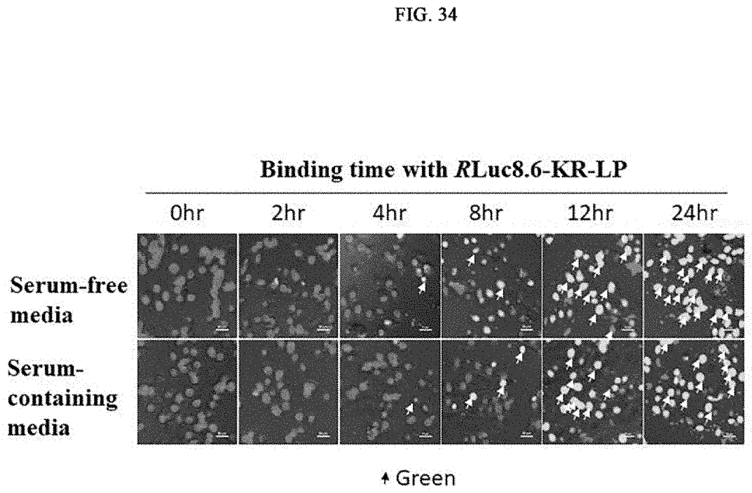

Referring to FIG. 34, it can be confirmed that, when cells were cultured in serum-free media or serum-containing media, in all cases, from an approximate 4-hour treatment time of RLuc8.6-KR-LP protein, the number of cells stained with SYTOX Green increased.

Accordingly, it can be demonstrated that, regardless of the presence of serum, the longer the reaction time after protein treatment, the higher the cell death rate.

### Example 16: Confirmation of Cell Death According to the Presence of Serum and Concentration of RLuc8.6-KR-LP Protein Using Co-h Substrate

MCF-7 cells were seeded at 5×10⁵ cells/mL in a 96-well plate (SPL Cell Culture Plate, 96 well (SPL, Cat #30096)), and cultured at 37° C. for 24 hours (overnight). After the culture for 24 hours, the cells were washed with FBS & Phenol red-free media (RPMI), and 100 μL of FBS & Phenol red-free media (RPMI) or FBS media (RPMI) containing a predetermined concentration of purified protein, followed by reaction at 37° C. for 12 hours.

After the reaction for 24 hours, the cells were washed with FBS & Phenol red-free media (RPMI), 100 μL of FBS & Phenol red-free media (RPMI) containing a Co-h substrate solution (final concentration: 150 μM) and SYTOX Green (final concentration: 261 nM) staining DNA of dead cells was added, followed by reaction at 37° C. for 30 minutes.

After the reaction, 10 μL of DAPI staining DNA of live cells was added, followed by reaction for 5 minutes. Cell death was confirmed by observing KillerRed, SYTOX Green and DAPI fluorescence using a confocal microscope. Cell death was observed with SYTOX Green (dead cell-specific dye, green) and DAPI (live cell-specific dye, blue) using a fluorescence microscope.

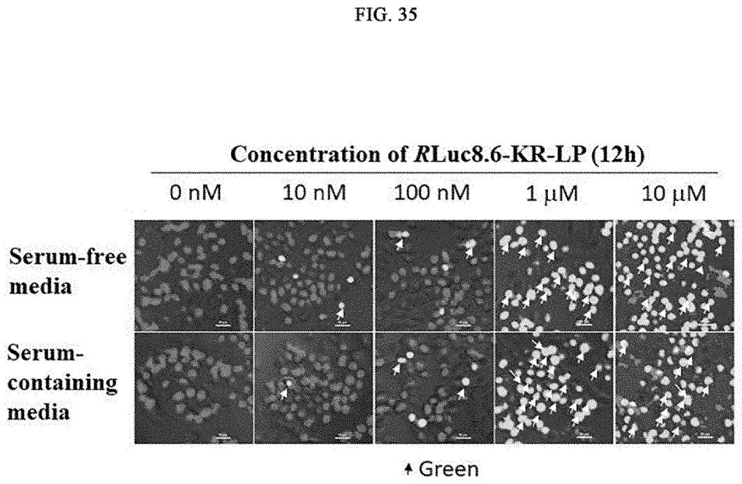

Referring to FIG. 35, it can be confirmed that, when cells were cultured in serum-free media or serum-containing media, in all cases, the higher the treated concentration of RLuc8.6-KR-LP, the higher the number of cells stained with SYTOX Green.

Accordingly, it can be demonstrated that, regardless of the presence of serum, the higher the concentration of the treated protein, the higher the cell death rate.

### Example 17: Flow Cytometric Analysis of KR, SYTOX Green and DAPI According to RLuc8.6-KR-LP Treatment for Cells and Reaction Time with Co-h Substrate

MCF-7 cells were seeded at 5×10⁵ cells/mL in a 96-well plate (SPL Cell Culture Plate, 96 well (SPL, Cat #30096)), and cultured at 37° C. for 24 hours (overnight). After the culture, the cells were washed with FBS & Phenol red-free media (RPMI), and 100 μL of FBS & Phenol red-free media (RPMI) containing a purified protein (final concentration: 10 μM), followed by reaction at 37° C. for 24 hours (overnight).

After washing with KillerRed: FBS & Phenol red-free media (RPMI), the cells were detached by treating 100 μL of Trypsin EDTA (Gibco™), and then centrifuged at 1,000 rpm for 3 minutes. The cells were filtered through a cell strainer (SPL, cat #93070) and resuspended in 400 μL of 1×DPBS containing 5% FBS. Specific cells exhibiting fluorescence in this solution were quantified using a flow cytometer (BD FACS Canto™)

The cells were washed with SYTOX Green: FBS & Phenol red-free media (RPMI), and 100 μL (final concentration: 150 μM) of FBS & Phenol red-free media (RPMI) containing a Co-h substrate solution was added, followed by incubation for 24 hours at 37° C. 50 μL of SYTOX Green (final concentration: 261 nM) staining DNA of dead cells was added without washing the cells, followed by reaction at 37° C. for 30 minutes. Without washing, the cells were detached by treatment with 100 μL of trypsin EDTA (Gibco™), and centrifuged at 1,000 rpm for 3 minutes. The cells were filtered through a cell strainer (SPL, cat #93070) and resuspended in 400 μL of 1×DPBS containing 5% FBS. Specific cells exhibiting fluorescence in this solution were quantified using Flow cytometer (BD FACS Canto™)

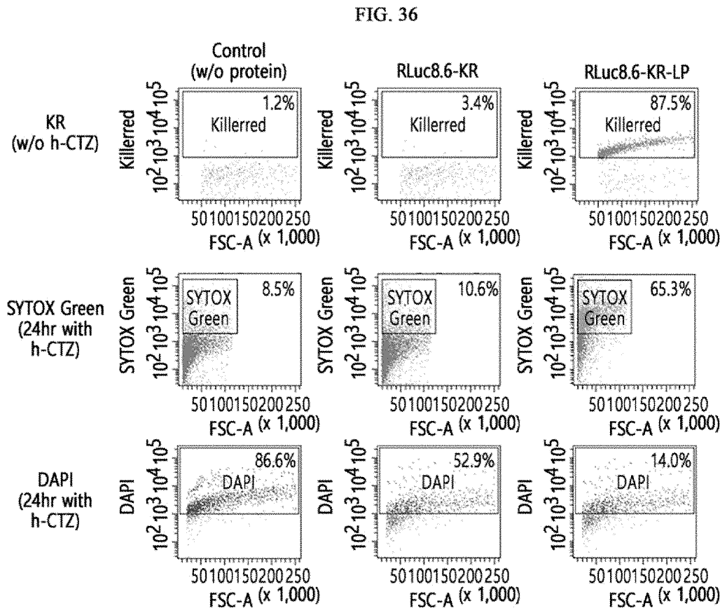

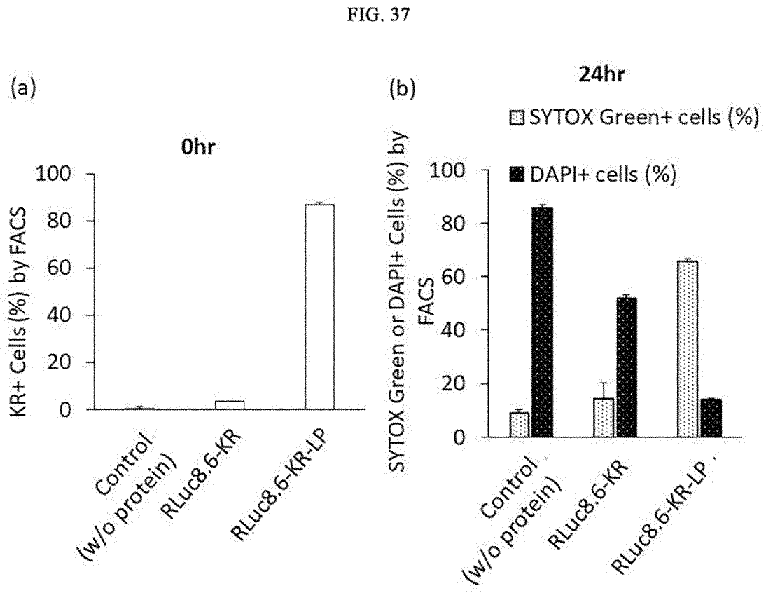

The cells were washed with DAPI: FBS & Phenol red-free media (RPMI), and 100 μL (final concentration: 150 μM) of FBS & Phenol red-free media (RPMI) containing a Co-h substrate solution was added, followed by incubation for 24 hours at 37° C. The cells were washed with FBS & Phenol red-free media (RPMI), and then 100 μL of media (RPMI) was added thereto. 10 μL of DAPI staining DNA of live cells was added, followed by reaction at 37° C. for 30 minutes. The cells were detached by treatment with 100 μL of trypsin EDTA (Gibco™) without washing, followed by centrifugation at 1,000 rpm for 3 minutes. The cells were filtered through a cell strainer (SPL, cat #93070) and resuspended in 400 μL of 1×DPBS containing 5% FBS. Specific cells exhibiting fluorescence in this solution were quantified using a flow cytometer (BD FACS Canto™) Referring to FIGS. 36 and 37, it can be confirmed that, in cells treated with

RLuc8.6-KR-LP protein, the number of cells exhibiting fluorescence of KillerRed increased and the number of cells exhibiting SYTOX Green increased, whereas the number of cells stained with DAPI decreased.

This is because the cells are treated with RLuc8.6-KR having no second protein, a lead peptide, serving to place proteins generating ROS as close as possible to cancer cells, and thus the number of cells stained with DAPI increases without a cytotoxic effect although there is a reaction with a substrate.

### Example 18: Confirmation of Cell Death after Light Irradiation According to the Presence of Lead Peptide and Cell Type

In various breast cancer cell lines (MCF-7, BT-474, MDA-MB-435, SK-BR-3, MDA-MB-231 and MCF-10A), the cytotoxic effect of RLuc8.6-KR-LP protein by light irradiation was confirmed.

Data on each breast cancer cell line is as follows (Table 4): MCF-7: (Origin: breast, mammary gland, Species: human—female, 69 years old, Caucasian, Growth pattern: monolayer, Media: RPMI1640 with L-glutamine (300 mg/L), 25 mM HEPES and 25 mM NaHCO₃, 90%; heat inactivated fetal bovine serum (FBS), 10%), purchased from Korean Cell Line Bank (KCLB).

SK-BR-7: (Origin: breast, mammary gland, Species: human—female, 43 years old, Caucasian, Growth pattern: monolayer, Media: RPMI1640 with L-glutamine (300 mg/L), 25 mM HEPES and 25 mM NaHCO₃, 90%; heat inactivated fetal bovine serum (FBS), 10%), purchased from Korean Cell Line Bank (KCLB).

MDA-MB-231: (Origin: breast, mammary gland, Species: human—female, 51 years old, Caucasian, Growth pattern: monolayer, Media: DMEM with glucose (4.5 g/L), L-glutamine and sodium pyruvate, 90%; heat inactivated fetal bovine serum (FBS), 10%), purchased from Korean Cell Line Bank (KCLB).

MDA-MB-435: (Origin: breast, mammary gland, Species: human—female, 31 years old, Caucasian, Media: DMEM with glucose (4.5 g/L), L-glutamine and sodium pyruvate, 90%; heat inactivated fetal bovine serum (FBS), 10%), purchased from ATCC (USA).

BT-474: (Origin: breast, mammary gland, Species: human, Media: RPMI1640 with L-glutamine (300 mg/L), 25 mM HEPES and 25 mM NaHCO₃, 90%; heat inactivated fetal bovine serum (FBS), 10%), purchased from Korean Cell Line Bank (KCLB).

MCF-10A: (Origin: breast, mammary gland, Species: human—female, 36 years old, Caucasian, Media: The base medium for this cell line (MEBM) with the additives can be obtained from Lonza/Clonetics Corporation as a kit: MEGM, Kit Catalog No. CC-3150), purchased from ATCC (USA).

TABLE 4

| | | | | | | | | | Response |
|---|---|---|---|---|---|---|---|---|---|
| Cell | Estrogen | Progesterone | HER2 | | | | | | to Luc- |
| line | receptor | receptor | receptor | CK5/6 | EGFR | Ki-67 | AR | Subtype | RGP-LP |
| MCF- | 6 | 6 | 0-1+ | − | 1+ | 90% | 7 | Luminal A | Yes |
| 7 | | | | | | | | | |
| BT- | 0 | 8 | 3+ | − | 1+ | 70% | 7 | Luminal B | No |
| 474 | | | | | | | | | |
| MDA- | 0 | 0 | 3+ | − | 0 | 80% | 6 | HER2 | Yes |
| MB- | | | | | | | | | |
| 435 | | | | | | | | | |
| SK- | 0 | 0 | 3+ | − | 2+ | 20% | 8 | HER2 | No |
| BR-3 | | | | | | | | | |
| MDA- | 0 | 0 | 0-1+ | − | 1+ | 100% | 8 | Basal | Yes |
| MB- | | | | | | | | | |
| 231 | | | | | | | | | |
| MCF- | 0 | 0 | 0-1+ | + | 2+ | 30% | 0 | Basal | No |
| 10A | | | | | | | | | |

The lead peptide (WLEAAYQRFL) used in this example is known to specifically bind to MCF-7, MDA-MB-231 and MDA-MB-435 cells.

The cells were seeded at 5×10⁵ cells/mL in 96-well plates (SPL Cell Culture Plate, 96 well (SPL, Cat #30096)), and cultured at 37° C. for 24 hours (overnight). After the culture for 24 hours, the cells were washed with FBS & phenol red-free media, and 100 μL of FBS & phenol red-free media containing a purified protein (final concentration: 10 μM) was added, followed by reaction at 37° C. for 24 hours. After the reaction for 24 hours, the cells were washed with FBS & phenol red-free media, and 100 μL of FBS & phenol red-free media was added.

After irradiation with light at 10 mW/cm² for 10 minutes, 50 μL (final concentration: 261 nM) of SYTOX Green staining DNA of dead cells was added, followed by reaction at 37° C. for 30 minutes. 10 μL of DAPI staining DNA of live cells was added, followed by reaction for 5 minutes. Cell death was confirmed by observing KillerRed, SYTOX Green and DAPI fluorescence using a confocal microscope.

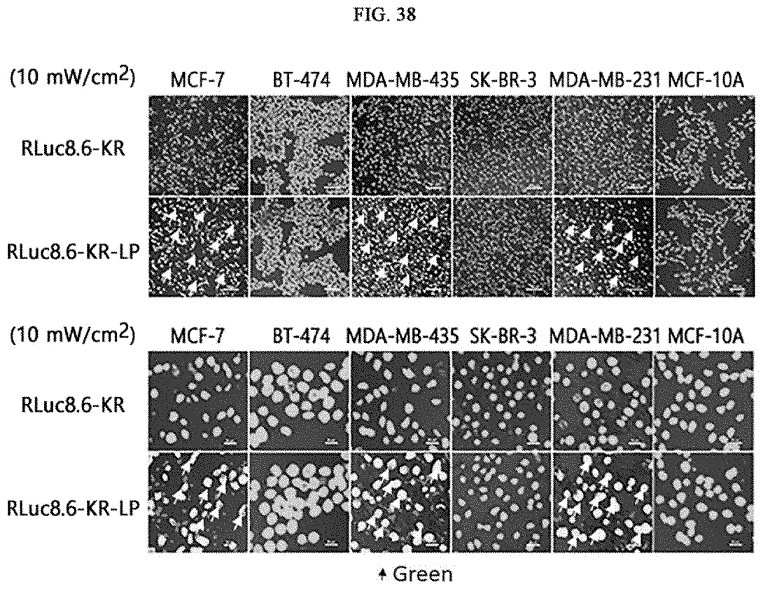

The result is shown in FIG. 38.

As a result, as in the known technical facts, it can be seen that the lead peptide of SEQ ID NO: 6 is specific for MCF-7, MDA-MB-231 and MDA-MB-435 cells. However, as shown in Table 4, the six breast cancer cell lines used in this example correspond to cell lines each having a characteristic of exhibiting different receptors. Furthermore, the MCF-7, MDA-MB-231 and MDA-MB-435 cancer cell lines commonly expressed different types of major receptors. Based on this fact, the inventors believed that the lead peptide of SEQ ID NO: 6 does not bind to the common receptor expressed by the MCF-7, MDA-MB-231 and MDA-MB-435 cancer cell lines, but bind to the common membrane protein of these cancer cells.

Therefore, with respect to the six experimental breast cancer cell lines, SYTOX Green staining can show that the RLuc8.6-KR-LP protein containing the lead peptide recognizes only the common membrane protein of the MCF-7, MDA-MB-231 and MDA-MB-435 cell lines, thereby killing the three types of cancer cell lines.

In addition, with respect to the six experimental breast cancer cell lines, DAPI staining can show that the RLuc8.6-KR protein without a lead peptide that can recognize the common membrane protein did not recognize the breast cancer cell lines in all experimental groups and thus any of the six breast cancer cell lines was not killed.

That is, as the LP of the RLuc8.6-KR-LP protein specifically binds to the membrane proteins of MCF-7, MDA-MB-231 and MDA-MB-435 cell lines, KR activated by external light irradiation provides ROS, resulting in the death of breast cancer cells.

### Example 19: Confirmation of Cell Death in the Presence or Absence of Substrate According to the Presence of Lead Peptide and Cell Type

The six breast cancer cell lines used in Example 22 were used.

The cells were seeded at 5×10⁵ cells/mL in 96-well plates (SPL Cell Culture Plate, 96 well (SPL, Cat #30096)), and cultured at 37° C. for 24 hours (overnight). After the culture for 24 hours, the cells were washed with FBS & phenol red-free media, and 100 μL of FBS & phenol red-free media containing a purified protein (final concentration: 10 μM) was added, followed by reaction at 37° C. for 24 hours.

Subsequently, the cells were washed with FBS & phenol red-free media, and 100 μL of FBS & phenol red-free media was added. 100 μL of FBS & Phenol red-free media (RPMI) containing a Co-h substrate solution (final concentration: 150 μM) was added, followed by reaction at 37° C. for 5 minutes.

After the reaction, 50 μL of SYTOX Green (final concentration: 261 nM) or 90 μL of ethidium homodimer (EthD-1) (final concentration 1×), which stains DNA of dead cells, was added, followed by reaction at 37° C. for 30 minutes. 10 μL of DAPI staining DNA of live cells was added, followed by reaction for 5 minutes. Cell death was confirmed by observing KillerRed, SYTOX Green, MiniSOG, EthD-1 and DAPI fluorescence using a confocal microscope.

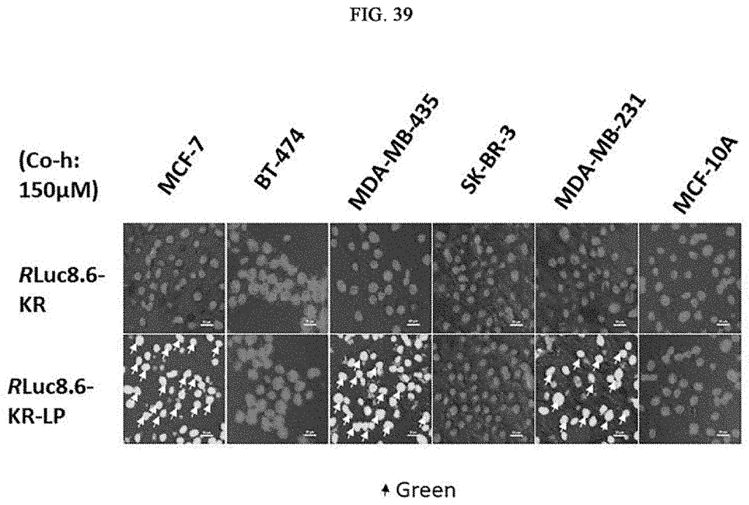

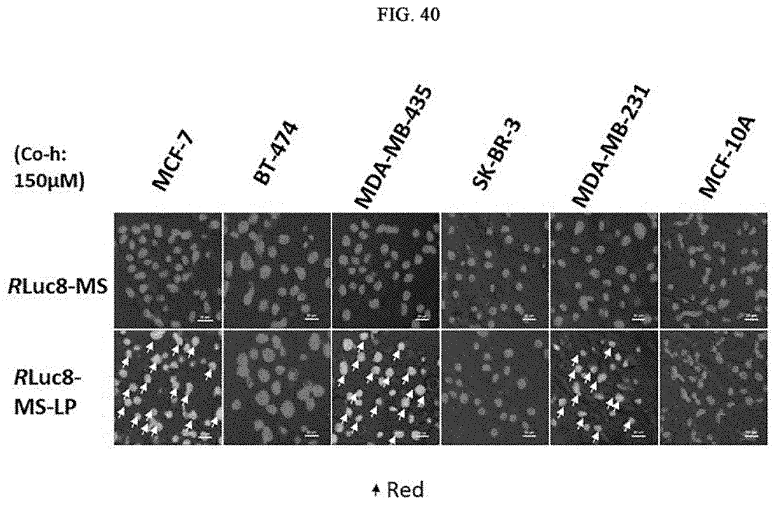

The result is shown in FIGS. 39 and 40. The result of this example could also be interpreted as having a similar meaning to that of Example 22.

That is, with respect to the six experimental breast cancer cell lines, SYTOX Green or EthD-1 staining can show that the RLuc8.6-KR-LP or RLuc8-MS-LP protein containing the lead peptide recognized only MCF-7, MDA-MB-231 and MDA-MB-435 cell lines and killed them.

In addition, DAPI staining can show that the RLuc8.6-KR or RLuc8-MS protein without a lead peptide did not recognize the breast cancer cell lines in all experimental groups, and thus none of the six breast cancer cell lines were killed.

That is, the LP of the RLuc8.6-KR-LP or RLuc8-MS-LP protein specifically binds to the common membrane protein of the MCF-7, MDA-MB-231 and MDA-MB-435 cell lines and the added substrate reacts with RLuc8 or RLuc8.6 protein to provide light so that KR or MS generates ROS to kill the breast cancer cells.

### Example 20: Cytotoxic Effect of RLuc8.6-KR-LP Protein by Bioluminescence in Patient Primary Cell-BL-067233

Cells were seeded at 5×10⁵ cells/mL in 96-well plates (SPL Cell Culture Plate, 96 well (SPL, Cat #30096)), and cultured at 37° C. for 24 hours. After the culture for 24 hours, the cells were washed with primary cell media, and 100 μL of primary cell media containing a purified protein (final concentration: 10 μM) was added, followed by reaction at 37° C. for 12 hours.

After the reaction for 24 hours, the cells were washed with primary cell media, and 100 μL of primary cell media was added. After treatment with a Co-h substrate solution (final concentration: 150 μM) for 5 minutes or irradiation with light at 10 mW/cm² for 5 minutes, 50 μL of SYTOX Green (final concentration: 261 nM) staining DNA of dead cells was added, followed by reaction at 37° C. for 30 minutes.

10 μL of DAPI staining DNA of live cells was added, followed by reaction for 5 minutes. Cell death was confirmed by fluorescence imaging of SYTOX Green and DAPI fluorescence according to the type of protein treating cells and the light irradiation time using a confocal microscope. Data on patient breast cancer cell lines used in this example are as follows (Table 5).

TABLE 5

| No. of | | | | | | |
|---|---|---|---|---|---|---|
| Patient | | | | | | |
| Primary | | | | Phenotype | Cytokeratin | |
| cell | Sex | Age | Stage | (ER, PR, Her2) | 5/6 | EGFR |
| 1 | Female | 32 | 3C | Triple negative | Positive | Positive |
| 2 | Female | 57 | 3B | Triple negative | Positive | Positive |
| 3 | Female | 48 | 2B | Triple negative | Positive | Positive |

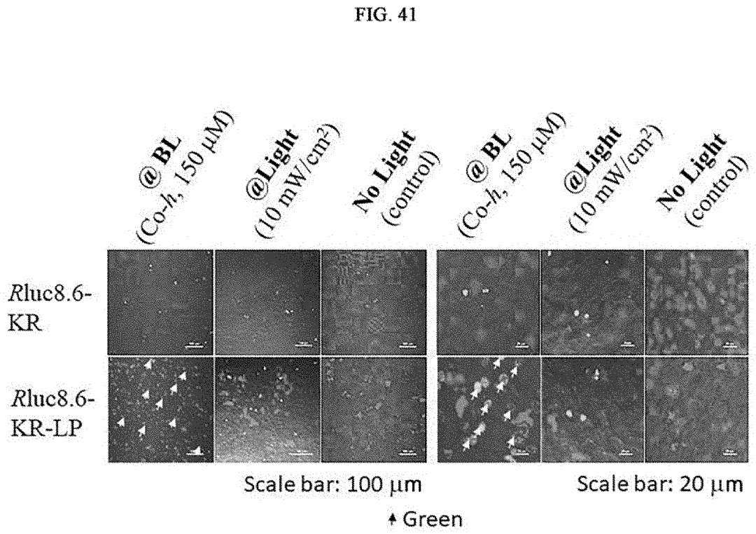

The result is shown in FIG. 41.

With respect to the patient breast cancer cell lines, in a breast cancer cell line treated with the lead peptide-containing RLuc8.6-KR-LP, a cytotoxic effect was exhibited. Here, the death of the Co-h substrate-added cells is more effectively shown.

Based on this result, the inventors could determine that the lead peptide used in this example also has specificity to the cancer patient-derived cancer cell line. Therefore, the cancer patient-derived cancer cell line is also expected to have the common membrane protein of the MCF-7, MDA-MB-231 and MDA-MB-435 cell lines. This shows that the lead peptide is able to target and kill corresponding cancer cells even when the cancer patient-derived cancer cell lines do not express representative cancer cell membrane receptors such as ER, PR and Her2 at all.

### Example 21: IVIS Spectrum in In Vivo Images and Measurement of Tissue Sizes According to RLuc8.6-KR-LP and Co-h Substrate Treated to Mice

MDA-MB-231 cancer cells (ATCC) were cultured in RPMI (Corning Inc.) containing 5% FBS (Corning Inc.) and 1% penicillin and streptomycin. Mice used in this experiment were 7-week-old NOD-SCID species, and purchased from Central Lab Animal Inc., and then the mice were raised without diet restriction for 2 weeks for acclimation at this research institute. The weight of a female mouse ranged from 17 to 23 g.

This animal experiment was conducted after approval by the Institute of Animal Care and Use Committee (IACUC) of the Asan Medical Center in compliance with the guidelines. 1×10⁶ cells/50 μL of MDA-MB-231 cells diluted in RPMI and 50 μL of Matrigel (Corning Inc.) were subcutaneously injected into the adipose body of a NOD-SCID mouse. On day 9 after cell injection, a protein containing 20 μL of Matrigel (RLuc8.6-KR-LP, final concentration: 50 μM) was injected into a tumor when the tumor size of the mouse was approximately 40 mm². After respiratory anesthesia of the mouse, fluorescence emitted from the protein was imaged using an IVIS spectrum (Xenogen Inc.). For BL-PDT, Co-h (5 μg/50 μL, Nanolight Technology.) was diluted in 1×PBS (Corning) and then subcutaneously injected. The light emission from Co-h was imaged after the IVIS spectrum was set to 5 seconds.

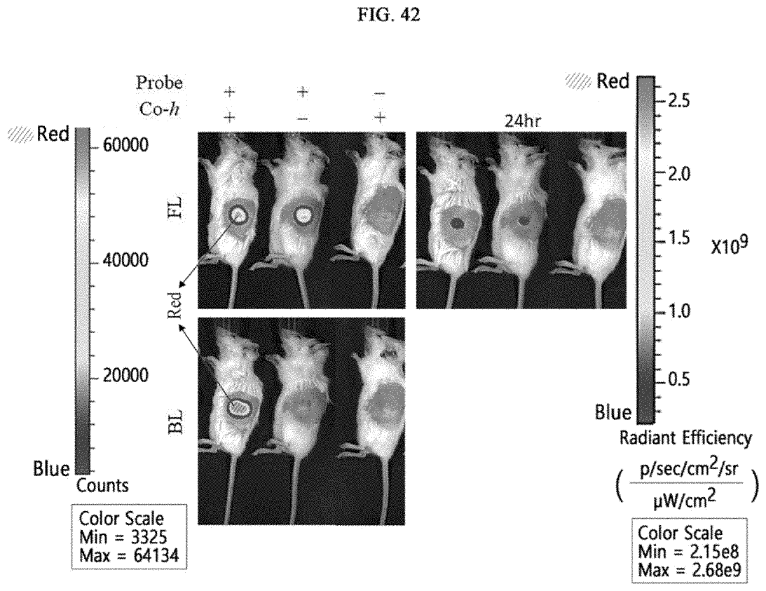

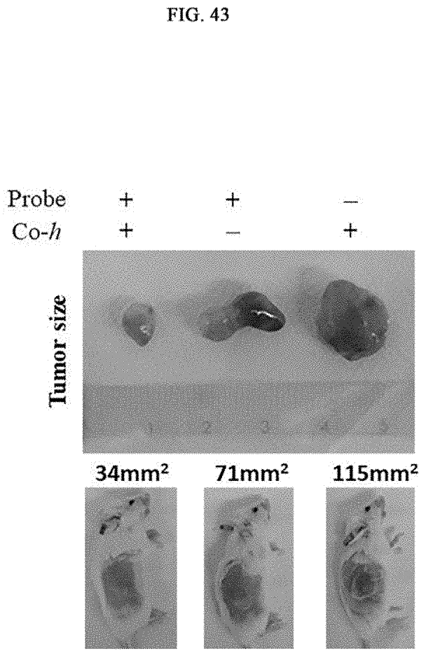

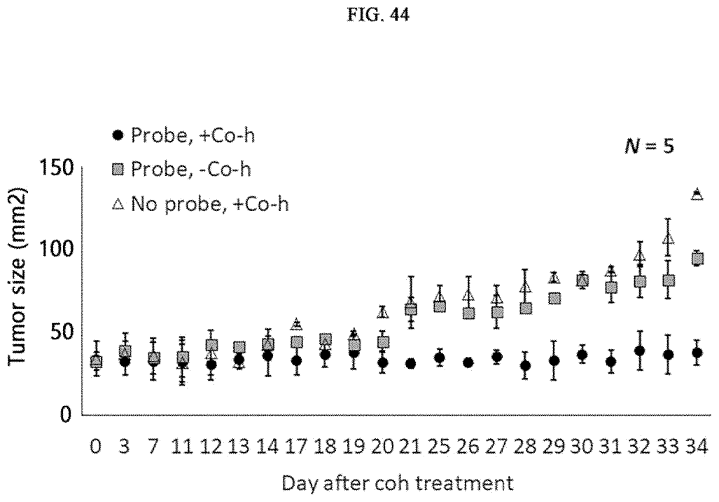

Referring to FIGS. 42 to 44, according to the IVIS spectrum, both the fluorescence and luminescence in tumors of the mice were confirmed, and it can be confirmed that the tumor size of the mice treated with RLuc8.6-KR-LP protein and a Co-h substrate solution was the smallest. The smallest tumor size means that the death of cancer cells occurred.

This suggests that, in the case of RLuc8.6-KR-LP protein, only in the presence of the Co-h substrate, the substrate reacts with RLuc8.6 to generate ROS, a second protein, a lead peptide, serving to place proteins generating the ROS as close as possible to cancer cells, moves these proteins to the cell membrane of the cancer cells, thereby providing the ROS to the cell membrane, and thus tumor growth is inhibited.

### INDUSTRIAL APPLICABILITY

The present application provides a composition for a cancer cell death, which targets a subject with a cancer disease, and a method for treating cancer.

As a preferable example, the composition and method can be used for a pharmaceutical composition and medicine for treating cancer.

[Sequence Listing Free Text]

SEQ ID NOs: 1 to 12 are protein sequences.

SEQ ID NOs: 13 to 28 are primer sequences.

1-22. (canceled)

23. A method for inducing a cancer cell death, comprising:
wherein the cancer cell death is that the cancer cell is killed by destroying the cell membrane of the cancer cell,
i) preparing a cancer cell death-fusion protein comprising:
a first protein for generating reactive oxygen species (ROS); wherein the first protein is one selected from KillerRed, MiniSOG, SOPP, FPFB, SuperNova, mKate2 and KillerOrange,
a second protein for specifically binding to a membrane protein constituting a cell membrane of the cancer cell; and
a third protein for providing a light; wherein the third protein comprises all or part of luciferase sequence,
ii) inducing the cancer cell death-fusion protein to be attached to the cell membrane of the cancer cell;
iii) attaching the cancer cell death-fusion protein to surface of the cell membrane, directly or indirectly without being introduced into an interior of the cancer cell;
iv) providing a light by the third protein so that the first protein of the cancer cell death fusion protein produce a ROS which is present on the cell membrane surface of the cancer cell; and
v) the ROS produced by the first protein acts on the cell membrane of the cancer cell, thereby destroying the cell membrane of the cancer cell to result in the cancer cell death.

24. The method of claim 23,
wherein the luciferase is one selected from Photobacteria luciferase, Firefly luciferase, Railroad worm luciferase, Renilla luciferase, Gaussia luciferase, Metridia luciferase, Cypridiana luciferase and Oplophorus luciferase (Nanoluc™).

25. The method of claim 23,
wherein the second protein is consisting of the amino acid sequence set forth in SEQ ID NO: 5.

26. The method of claim 23,
wherein providing a light by the third protein is performed by providing a specific substrate corresponding to the third protein.

27. The method of claim 26,
wherein the specific substrate is one selected form a luciferin and luciferin variant.

28. The method of claim 27,
wherein the luciferin variant is one selected form a coelenterazine and coelenterazine derivative.

29. The method of claim 23,
wherein the cancer cell death-fusion protein further comprises at least one of a first linker capable of liking the first protein with the second protein; or a second linker capable of liking the second protein with the third protein.

30. The method of claim 23,
wherein the ROS is one or more selected from superoxide, hydroxyl radical, singlet oxygen, hydrogen peroxide and hypochlorous acid.

31. The method of claim 23,
wherein the cancer cell is one selected from skin cancer cell, breast cancer cell, uterine cancer cell, lung cancer cell, liver cancer cell, gastric cancer cell, colon cancer cell, pancreatic cancer cell and blood cancer cell.

32. A method for treating cancer, comprising,
i) administering a cancer cell death-fusion protein to a subject;
wherein the cancer cell death-fusion protein comprising:
a first protein for generating reactive oxygen species (ROS); wherein the first protein is one selected from KillerRed, MiniSOG, SOPP, FPFB, SuperNova, mKate2 and KillerOrange,
a second protein for specifically binding to a membrane protein constituting a cell membrane of the cancer cell; and
a third protein for providing a light; wherein the third protein comprises all or part of luciferase sequence,
ii) providing a light to produce ROS by the first protein;
wherein the second protein selectively recognizes only a cancer cell and binds to a membrane protein constituting a cell membrane of the cancer cell, and
the ROS produced by the activation of the first protein by a light, is provided to the cell membrane of the cancer cell, thereby destroying the cell membrane of the cancer cell to result in the cancer cell death.

33. The method of claim 32,
wherein the luciferase is one selected from Photobacteria luciferase, Firefly luciferase, Railroad worm luciferase, Renilla luciferase, Gaussia luciferase, Metridia luciferase, Cypridiana luciferase and Oplophorus luciferase (Nanoluc™).

34. The method of claim 32,
wherein the method comprises further comprises providing a specific substrate corresponding to the third protein.

35. The method of claim 34,
wherein the specific substrate is one selected form a luciferin and luciferin variant.

36. The method of claim 35,
wherein the luciferin variant is one selected form a coelenterazine and coelenterazine derivative.

37. The method of claim 32,
wherein the administering is carried out by one or more method selected from oral administration, intraperitoneal administration, intravenous administration, intramuscular administration, subcutaneous administration, endothelial administration, intranasal administration, intrapulmonary administration, intratumor administration, rectal administration, intracavitary administration and intrathecal administration.

38. The method of claim 32,
wherein the cancer cell death-fusion protein further comprises at least one of a first linker capable of liking the first protein with the second protein; or a second linker capable of liking the second protein with the third protein.

39. The method of claim 32,
wherein the cancer is one selected from skin cancer, breast cancer, uterine cancer, lung cancer, liver cancer, gastric cancer, colon cancer, pancreatic cancer cell and blood cancer.

40. The method of claim 32,
wherein the second protein is consisting of the amino acid sequence set forth in SEQ ID NO: 5.

41. The method of claim 32,
wherein the ROS is one or more selected from superoxide, hydroxyl radical, singlet oxygen, hydrogen peroxide and hypochlorous acid.


SEQUENCE LISTING (28 sequences)

SEQ ID NO: 1 - protein, 257 aa, Artificial Sequence
  KillerRed
MLCCMRRTKQVEKNDEDQKISEGGPALFQSDMTFKIFIDGEVNGQKFTIVADGSSKFPHGDFNVHAVCETGKLPMSWKPICHLIQYGEPFFARYPDGISHFAQECFPEGLSIDRTVRFENDGTMTSHHTYELDDTCVVSRITVNCDGFQPDGPIMRDQLVDILPNETHMFPHGPNAVRQLAFIGFTTADGGLMMGHFDSKMTFNGSRAIEIPGPHFVTIITKQMRDTSDKRDHVCQREVAYAHSVPRITSAIGSDED

SEQ ID NO: 2 - protein, 106 aa, Artificial Sequence
  MiniSOG
MEKSFVITDPRLPDNPIIFASDGFLELTEYSREEILGRNGRFLQGPETDQATVQKIRDAIRDQREITVQLINYTKSGKKFWNLLHLQPMRDQKGELQYFIGVQLDG

SEQ ID NO: 3 - protein, 311 aa, Artificial Sequence
  RLuc8
MASKVYDPEQRKRMITGPQWWARCKQMNVLDSFINYYDSEKHAENAVIFLHGNATSSYLWRHVVPHIEPVARCIIPDLIGMGKSGKSGNGSYRLLDHYKYLTAWFELLNLPKKIIFVGHDWGAALAFHYAYEHQDRIKAIVHMESVVDVIESWDEWPDIEEDIALIKSEEGEKMVLENNFFVETVLPSKIMRKLEPEEFAAYLEPFKEKGEVRRPTLSWPREIPLVKGGKPDVVQIVRNYNAYLRASDDLPKLFIESDPGFFSNAIVEGAKKFPNTEFVKVKGLHFLQEDAPDEMGKYIKSFVERVLKNEQ

SEQ ID NO: 4 - protein, 311 aa, Artificial Sequence
  RLuc8.6
MASKVYDPEQRKRMITGPQWWARCKQMNVLDSFINYYDSEKHAENAVIFLHGNATSSYLWRHVVPHIEPVARCIIPDLIGMGKSGKSGNGSYRLLDHYKYLTAWFELLNLPKKIIFVGHDWGSALAFHYAYEHQDRIKAIVHMESVVDVIESWMGWPDIEEELALIKSEEGEKMVLENNFFVETLLPSKIMRKLEPEEFAAYLEPFKEKGEVRRPTLSWPREIPLVKGGKPDVVQIVRNYNAYLRASDDLPKLFIESDPGFFYNAIVEGAKKFPNTEFVKVKGLHFLQEDAPDEMGKYIKSFVERVLKNEQ

SEQ ID NO: 5 - protein, 10 aa, Artificial Sequence
  Lead Peptide
  misc_feature (2)..(2): Xaa may be any one selected from Ala, Arg, Asn, Asp, Cys, Gln, Glu, Gly, His, Ile, Leu, Lys, Met, Phe, Pro, Ser, Thr, Trp, Tyr, and Val
WXEAAYQRFL

SEQ ID NO: 6 - protein, 10 aa, Artificial Sequence
  Lead Peptide
WLEAAYQRFL

SEQ ID NO: 7 - protein, 290 aa, Artificial Sequence
  pRSET-KillerRed
MRGSHHHHHHGMASMTGGQQMGRDLYDDDDKDPMLCCMRRTKQVEKNDEDQKISEGGPALFQSDMTFKIFIDGEVNGQKFTIVADGSSKFPHGDFNVHAVCETGKLPMSWKPICHLIQYGEPFFARYPDGISHFAQECFPEGLSIDRTVRFENDGTMTSHHTYELDDTCVVSRITVNCDGFQPDGPIMRDQLVDILPNETHMFPHGPNAVRQLAFIGFTTADGGLMMGHFDSKMTFNGSRAIEIPGPHFVTIITKQMRDTSDKRDHVCQREVAYAHSVPRITSAIGSDED

SEQ ID NO: 8 - protein, 606 aa, Artificial Sequence
  pRSET-RLuc8.6-KillerRed
MRGSHHHHHHGMASMTGGQQMGRDLYDDDDKDPMASKVYDPEQRKRMITGPQWWARCKQMNVLDSFINYYDSEKHAENAVIFLHGNATSSYLWRHVVPHIEPVARCIIPDLIGMGKSGKSGNGSYRLLDHYKYLTAWFELLNLPKKIIFVGHDWGSALAFHYAYEHQDRIKAIVHMESVVDVIESWMGWPDIEEELALIKSEEGEKMVLENNFFVETLLPSKIMRKLEPEEFAAYLEPFKEKGEVRRPTLSWPREIPLVKGGKPDVVQIVRNYNAYLRASDDLPKLFIESDPGFFYNAIVEGAKKFPNTEFVKVKGLHFLQEDAPDEMGKYIKSFVERVLKNEQEFGGGMLCCMRRTKQVEKNDEDQKISEGGPALFQSDMTFKIFIDGEVNGQKFTIVADGSSKFPHGDFNVHAVCETGKLPMSWKPICHLIQYGEPFFARYPDGISHFAQECFPEGLSIDRTVRFENDGTMTSHHTYELDDTCVVSRITVNCDGFQPDGPIMRDQLVDILPNETHMFPHGPNAVRQLAFIGFTTADGGLMMGHFDSKMTFNGSRAIEIPGPHFVTIITKQMRDTSDKRDHVCQREVAYAHSVPRITSAIGSDED

SEQ ID NO: 9 - protein, 619 aa, Artificial Sequence
  pRSET-RLuc8.6-KillerRed-Lead peptide
MRGSHHHHHHGMASMTGGQQMGRDLYDDDDKDPMASKVYDPEQRKRMITGPQWWARCKQMNVLDSFINYYDSEKHAENAVIFLHGNATSSYLWRHVVPHIEPVARCIIPDLIGMGKSGKSGNGSYRLLDHYKYLTAWFELLNLPKKIIFVGHDWGSALAFHYAYEHQDRIKAIVHMESVVDVIESWMGWPDIEEELALIKSEEGEKMVLENNFFVETLLPSKIMRKLEPEEFAAYLEPFKEKGEVRRPTLSWPREIPLVKGGKPDVVQIVRNYNAYLRASDDLPKLFIESDPGFFYNAIVEGAKKFPNTEFVKVKGLHFLQEDAPDEMGKYIKSFVERVLKNEQEFGGGMLCCMRRTKQVEKNDEDQKISEGGPALFQSDMTFKIFIDGEVNGQKFTIVADGSSKFPHGDFNVHAVCETGKLPMSWKPICHLIQYGEPFFARYPDGISHFAQECFPEGLSIDRTVRFENDGTMTSHHTYELDDTCVVSRITVNCDGFQPDGPIMRDQLVDILPNETHMFPHGPNAVRQLAFIGFTTADGGLMMGHFDSKMTFNGSRAIEIPGPHFVTIITKQMRDTSDKRDHVCQREVAYAHSVPRITSAIGSDEDGGGWLEAAYQRFL

SEQ ID NO: 10 - protein, 139 aa, Artificial Sequence
  pRSET-MiniSOG
MRGSHHHHHHGMASMTGGQQMGRDLYDDDDKDPMEKSFVITDPRLPDNPIIFASDGFLELTEYSREEILGRNGRFLQGPETDQATVQKIRDAIRDQREITVQLINYTKSGKKFWNLLHLQPMRDQKGELQYFIGVQLDG

SEQ ID NO: 11 - protein, 455 aa, Artificial Sequence
  pRSET-RLuc8-MiniSOG
MRGSHHHHHHGMASMTGGQQMGRDLYDDDDKDPMASKVYDPEQRKRMITGPQWWARCKQMNVLDSFINYYDSEKHAENAVIFLHGNATSSYLWRHVVPHIEPVARCIIPDLIGMGKSGKSGNGSYRLLDHYKYLTAWFELLNLPKKIIFVGHDWGAALAFHYAYEHQDRIKAIVHMESVVDVIESWDEWPDIEEDIALIKSEEGEKMVLENNFFVETVLPSKIMRKLEPEEFAAYLEPFKEKGEVRRPTLSWPREIPLVKGGKPDVVQIVRNYNAYLRASDDLPKLFIESDPGFFSNAIVEGAKKFPNTEFVKVKGLHFLQEDAPDEMGKYIKSFVERVLKNEQEFGGGMEKSFVITDPRLPDNPIIFASDGFLELTEYSREEILGRNGRFLQGPETDQATVQKIRDAIRDQREITVQLINYTKSGKKFWNLLHLQPMRDQKGELQYFIGVQLDG

SEQ ID NO: 12 - protein, 468 aa, Artificial Sequence
  pRSET-RLuc8-MiniSOG-Lead Peptide
MRGSHHHHHHGMASMTGGQQMGRDLYDDDDKDPMASKVYDPEQRKRMITGPQWWARCKQMNVLDSFINYYDSEKHAENAVIFLHGNATSSYLWRHVVPHIEPVARCIIPDLIGMGKSGKSGNGSYRLLDHYKYLTAWFELLNLPKKIIFVGHDWGAALAFHYAYEHQDRIKAIVHMESVVDVIESWDEWPDIEEDIALIKSEEGEKMVLENNFFVETVLPSKIMRKLEPEEFAAYLEPFKEKGEVRRPTLSWPREIPLVKGGKPDVVQIVRNYNAYLRASDDLPKLFIESDPGFFSNAIVEGAKKFPNTEFVKVKGLHFLQEDAPDEMGKYIKSFVERVLKNEQEFGGGMEKSFVITDPRLPDNPIIFASDGFLELTEYSREEILGRNGRFLQGPETDQATVQKIRDAIRDQREITVQLINYTKSGKKFWNLLHLQPMRDQKGELQYFIGVQLDGGGGWLEAAYQRFL

SEQ ID NO: 13 - DNA, 32 bp, Artificial Sequence
  pRSET-KillerRed)F
cgggatcccatgctgtgctgtatgagaagaac

SEQ ID NO: 14 - DNA, 34 bp, Artificial Sequence
  pRSET-KillerRed)R
cccaagcttctaatcctcgtcgctaccgatggcg

SEQ ID NO: 15 - DNA, 37 bp, Artificial Sequence
  pRSET-RLuc8.6-KillerRed)F
cggaattcggaggaggaatgctgtgctgtatgagaag

SEQ ID NO: 16 - DNA, 31 bp, Artificial Sequence
  pRSET-RLuc8.6-KillerRed)R
cccaagcttctaatcctcgtcgctaccgatg

SEQ ID NO: 17 - DNA, 37 bp, Artificial Sequence
  pRSET-RLuc8.6-KillerRed-Lead peptide)F
cggaattcatgggaggaggactgtgctgtatgagaag

SEQ ID NO: 18 - DNA, 28 bp, Artificial Sequence
  pRSET-RLuc8.6-KillerRed-Lead peptide)R1
cggcctccagccatcctcctccatcctc

SEQ ID NO: 19 - DNA, 28 bp, Artificial Sequence
  pRSET-RLuc8.6-KillerRed-Lead peptide)R2
aagcgctggtaggcggcctccagccatc

SEQ ID NO: 20 - DNA, 31 bp, Artificial Sequence
  pRSET-RLuc8.6-KillerRed-Lead peptide)R3
cccaagcttctacaggaagcgctggtaggcg

SEQ ID NO: 21 - DNA, 32 bp, Artificial Sequence
  pRSET-MiniSOG)F
cgggatcccatggagaagagcttcgtgatcac

SEQ ID NO: 22 - DNA, 28 bp, Artificial Sequence
  pRSET-MiniSOG)R
cccaagcttctagccgtccagctgcacg

SEQ ID NO: 23 - DNA, 35 bp, Artificial Sequence
  pRSET-RLuc8-MiniSOG)F
cggaattcggaggaggaatggagaagagcttcgtg

SEQ ID NO: 24 - DNA, 28 bp, Artificial Sequence
  pRSET-RLuc8-MiniSOG)R
cccaagcttctagccgtccagctgcacg

SEQ ID NO: 25 - DNA, 37 bp, Artificial Sequence; identical to SEQ ID NO: 17
  pRSET-RLuc8-MiniSOG-Lead peptide)F

SEQ ID NO: 26 - DNA, 25 bp, Artificial Sequence
  pRSET-RLuc8-MiniSOG-Lead peptide)R1
ggcctccagccatcctcctccgccg

SEQ ID NO: 27 - DNA, 27 bp, Artificial Sequence
  pRSET-RLuc8-MiniSOG-Lead peptide)R2
agcgctggtaggcggcctccagccatc

SEQ ID NO: 28 - DNA, 31 bp, Artificial Sequence; identical to SEQ ID NO: 20
  pRSET-RLuc8-MiniSOG-Lead peptide)R3